(19) United States
(12) Patent Application Publication
(10) Pub. No.: US 2007/0190142 A1
(43) Pub. Date: Aug. 16, 2007

(54) Dosage forms for the delivery of drugs of abuse and related methods

(75) Inventors: Jorg BREITENBACH, Mannheim (DE); Ute Lander, Dannenfels (DE); Jorg Rosenberg, Ellerstadt (DE); Markus Maegerlein, Mannheim (DE); Gerd Woehrle, Eppelheim (DE)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(73) Assignee: Abbott GmbH & Co. KG, Wiesbaden (DE)

(21) Appl. No.: 11/625,705

(22) Filed: Jan. 22, 2007

Related U.S. Application Data
(60) Provisional application No. 60/760,707, filed on Jan. 21, 2006.

(51) Int. Cl. A61K 9/26

(52) U.S. Cl. 424/469

(57) ABSTRACT

A dosage form and method for the delivery of drugs, particularly drugs of abuse, characterized by resistance to solvent extraction, tampering, crushing, or grinding, and providing an initial burst of release of drug followed by a prolonged period of controllable drug release.

### TECHNICAL FIELD OF THE INVENTION

The present invention relates to compositions for oral administration. The present invention preferably comprises at least one abuse-resistant drug delivery composition for delivering a drug having abuse potential, related methods of preparing these dosage forms, and methods of treating a patient in need thereof comprising administering the inventive compositions to the patient.

### BACKGROUND OF THE INVENTION

Abuse of prescription drugs has become a public health problem in many communities. One common class of drugs that is subject to abuse is the opioid class. Opioids are the major class of analgesics used in the management of moderate to severe pain in the United States of America because of their effectiveness, ease of titration, and favorable risk-to-benefit ratio.

One of the effects of opioid administration is the ability of such drugs in some individuals to alter mood and feeling in a manner so as to provide a desirable sense of “well-being” dissociated from therapeutic ameliorative effects. This mood-altering effect is found by some individuals to be extremely pleasurable, and may be related to the fact that some users are at high risk of using the drugs illicitly and becoming addicted to opioids.

Three basic patterns of opioid abuse have been identified in the United States. One involves individuals whose drug use begins in the context of medical treatment and initially obtain their drug through medical channels. Another involves persons who begin their drug use with experimental or “recreational” drug use and progress to more intensive drug use. Lastly, there are users who begin using drugs obtained from medical channels or through recreational drug channels, but later switch to oral opioids obtained from organized addiction treatment programs.

Abuse of opioids by the oral route is significant. However, another significant problem for opioid abuse appears to be the abuse of the drugs by parenteral administration, particularly by injection. Rapid injection of opioid agonists is known to produce a warm flushing of the skin and sensations. The state, known alternatively as a “rush,” “kick,” or “thrill,” typically lasts for only about 45 seconds but is found extremely pleasurable to addicts. Addicted individuals will extract solid dosage forms of opioids and then inject the same to attain such a state. Opioids have also been known to be abused via nasal administration, where the potential drug of abuse is crushed and powdered and snorted nasally.

Some presently proposed pharmacological methods for dissuading the extraction of oral opioids incorporate of one or more of opioid antagonists, mixed opioid agonist-antagonists and other adversative drug agents, with the therapeutic opioid agonist. In most proposed systems, the dose of opioid antagonist is not orally active but will block the effects desired by abusers of the agonist drug, or mixed agonist-antagonist drug, when the drug is dissolved to obtain the agonist (or mixed agonist-antagonist drug) and the opioid is subsequently administered parenterally. In these cases, however, physicians may be concerned that inappropriate release of aversive drugs may cause harm and some have expressed a reluctance to prescribe opioids co-formulated with aversive agents.

For example, a drawback of approaches that incorporate opioid antagonists into the opioid preparation to dissuade abuse is that opioid antagonists themselves have side effects that may be disadvantageous. For example, nalorphine causes unpleasant reactions such as anxiety, irrational feelings, hallucinations, respiratory depression and miosis. Seizures have been reported with naloxone, albeit infrequently, and in postoperative patients, pulmonary edema and ventricular fibrillation have been seen with high dosages. Naltrexone has been reported to have the capacity to cause hepatocellular injury when given in doses as low as fivefold or less of therapeutic doses. Nalmefene, although usually well tolerated, has been reported to cause nausea, vomiting and tachycardia in some individuals. Small doses of any of these opioid antagonists can also precipitate withdrawal in opioid addicted individuals even at low doses, a phenomenon that can be extremely dangerous depending upon where the addicted individual takes the drug.

Similarly to the opioids, many other classes of drugs are also subject to abuse, although the patterns and effects of the abuse differ to some degree.

WO 2005/079760 (Euroceltique) discloses melt-extruded, multiparticulated, controlled release formulations containing a neutral poly(ethyl acrylate, methyl methacrylate) copolymer and an active ingredient. The formulations are said to show rubber-like properties such that they exhibit enhanced resistance to tampering.

US 2003/0118641 (Boehringer Ingelheim) relates to a method for reducing the abuse potential of an oral dosage form of an opioid extractable by commonly available household solvents said method comprising combining a therapeutically effective amount of the opioid compound, a matrix-forming polymer and an ionic exchange resin. Preference is given to ionic exchange resins that are strongly acidic.

WO 00/041481 (Knoll) relates to medicament forms containing active substances with high water-solubility in a matrix based on acrylate polymers.

US Patent Application Publication No. 2006/0002860 (Bartholomaus et al.) relates to tamper-resistant drug formulations useful in the context of drugs of abuse.

While numerous compositions, formulations and methodologies exist to address abuse of drugs, all compositions, formulations and methods have limitations to a greater or lesser extent. Accordingly, there is a need for providing new and/or improved formulations, compositions and methods of preventing abuse of drugs having abuse potential.

This background information is provided for the purpose of making known some information believed by the applicant to be of possible relevance to the present invention. No admission is intended, nor should be construed, that any of the preceding information constitutes prior art to the present invention.

### SUMMARY OF THE INVENTION

Certain preferred embodiments of the present invention provide dosage forms and methods for the delivery of drugs, particularly drugs of abuse, characterized by resistance to solvent extraction; tampering, crushing or grinding, and providing an initial burst of release of drug followed by a prolonged period of controllable drug release.

One exemplary embodiment of the present invention provides an abuse-deterrent drug formulation comprising a melt-processed mixture of: a) at least one abuse-relevant drug, b) at least one cellulose ether or cellulose ester, and c) at least one alkyl alkacrylate polymer, alkacrylate polymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily.

Another exemplary embodiment of the present invention provides a monolithic, sustained release oral dosage formulation comprising a melt-processed mixture of: a) an analgesically effective amount of at least one an abuse-relevant drug, b) at least one cellulose ether or cellulose ester, and c) at least one alkyl alkacrylate polymer, alkacrylate polymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted for sustained release so as to be useful for oral administration to a human 3, 2, or 1 times daily.

Yet another exemplary embodiment of the present invention provides an oral sustained release dosage formulation of a drug characterized by at least two of the following features: a) the drug that is extracted from the formulation by 40% aqueous ethanol within one hour at 37° C. is less than or equal twice the amount of the drug that is extracted by 0.01 N hydrochloric acid within one hour at 37° C., b) the formulation does not break under a force of 150 newtons, preferably 300 newtons, more preferably 450 newtons, yet more preferably 500 newtons as measured by “Pharma Test PTB 501” hardness tester, and c) the formulation releases at least 15% of the one drug and not more than 45% of the one drug during the first hour in vitro dissolution testing and preferably also in vivo.

Another exemplary embodiment of the present invention provides a non-milled, melt-extruded drug formulation comprising a drug with abuse potential.

An exemplary embodiment of the present invention also provides a monolithic, non-milled, non-multiparticulated, melt-extruded drug formulation comprising a drug with abuse potential having a diameter from about at least 5.1 mm to about 10 mm and a length from about 5.1 mm to about 30 mm.

Another exemplary embodiment of the present invention provides a process for the manufacture of an abuse-resistant drug dosage formulation comprising melt extruding a formulation comprising at least one therapeutic drug further comprising directly shaping the extrudate into a dosage form without (an intermediate) milling step or multiparticulating step.

Yet another exemplary embodiment of the present invention provides a monolithic, non-milled, melt-extruded drug formulation comprising a drug with abuse potential wherein the monolithic formulation has a substantially similar drug release profile to a crushed form of the monolithic formulation wherein the monolithic formulation is crushed at about 20,000 rpm to about 50,000 rpm in a coffee grinding machine for about 60 seconds in a grinder having stainless steel blades, about a 150 watt motor, and a capacity for about 90 milliliters (i.e., about 3 ounces) of coffee beans.

Another exemplary embodiment of the present invention provides an abuse-deterrent drug formulation comprising a melt-processed mixture of: a) at least one abuse-relevant drug, b) at least one rate altering pharmaceutically acceptable polymer, copolymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and

the drug formulation is adapted so as to be useful for oral administration to a human 3, 2, or 1 times daily.

Yet another exemplary embodiment of the present invention provides an abuse-deterrent drug formulation comprising a melt-processed mixture of: a) at least one abuse-relevant drug, wherein said drug is hydrocodone (or a pharmaceutically accepted salt like e.g. hydrocodone bitartrate pentahemihydrate), b) at least one cellulose ether or cellulose ester, and c) at least one acrylic polymer, methacrylic polymer, or a combination thereof. In this embodiment, the drug formulation is adapted so as to be useful for oral administration to a human 3, 2, or 1 times daily; and about ninety percent of the hydrocodone is released in vitro at about 4-6 hours when adapted to be administered 3 times a day, at about 6-10 hours when adapted to be administered 2 times a day and about 16-22 hours when adapted to be administered 1 time a day.

Another exemplary embodiment of the present invention also provides an abuse-deterrent drug formulation comprising a melt-processed mixture of: a) at least one opioid; and b) at least one rate altering pharmaceutically acceptable polymer, copolymer, or a combination thereof. In this embodiment, the amount of the drug that is extracted from the formulation by 40% aqueous ethanol within one hour at 37° C. is about 70% to about 110% of the amount of the drug that is extracted by 0.01 N hydrochloric acid within one hour at 37° C.; and the drug formulation is adapted so as to be useful for oral administration to a human 3, 2, or 1 times daily. This and other embodiments have desirable pharmacokinetic profiles.

In another exemplary embodiment, the present invention provides a method for treating pain in a human patient, comprising orally administering to the human patient a formulation from any one of the above embodiments.

These and other objects, advantages, and features of the invention will become apparent to those persons skilled in the art upon reading the details of the methods of the invention and compositions used therein as more fully described below.

### BRIEF DESCRIPTION OF THE DRAWINGS

FIG. 1 depicts the rate of dissolution of various drug dosage forms 1-6 in 0.01 M hydrochloric acid.

FIG. 2 depicts the rate of dissolution of various drug dosage forms 1-6 in 20% aqueous ethanol.

FIG. 3 depicts the rate of dissolution of various drug dosage forms 7-9 of hydrocodone in 0.01 N hydrochloric acid.

FIG. 4 depicts rate of dissolution of various drug dosage forms 7-9 of acetaminophen (APAP; also known as paracetamol) in 0.01 N hydrochloric acid.

FIG. 5 depicts the rate of dissolution of various drug dosage forms 7-9 of hydrocodone in 40% aqueous ethanol.

FIG. 6 depicts rate of dissolution of various drug dosage forms 7-9 of acetaminophen (APAP) in 40% aqueous ethanol.

FIG. 7 depicts a force transducer and an exemplary tablet holder having a tablet used for measuring breaking strength of tablets.

FIG. 8 depicts a cylinder with a wedge-shaped tip having certain exemplary dimensions useful for conducting “Pharma Test PTB 501” for measuring hardness of a tablet.

FIG. 9 (A) depicts the chemical structure for acetaminophen (APAP), (B) depicts half-life, Cmax, Tmax and AUC for some embodiments of the inventive formulation (30) following oral dose administration of this formulation (30) in male minipigs Goettingen) (C) depicts mean (±SEM) plasma concentrations of acetaminophen following oral dose administration of an embodiment of the inventive formulation (30) in male minipigs (Goettingen).

FIG. 10 (A) depicts half-life, Cmax, Tmax and AUC for certain embodiments of the inventive formulation (Forms 26, 27, 28, 29, 30), Control 1 and Control 2 in male minipigs (Goettingen) and Control 1 formulation in human (B) depicts mean (±SEM) plasma concentrations of acetaminophen following oral dose administration of certain embodiments of the inventive formulation (Forms 26, 27, 28, 29, 30), control 1 and control 2 in male minipigs (Goettingen) and Control 1 formulation in human.

FIG. 11 depicts mean (±SEM) plasma concentrations of acetaminophen following oral dose administration of certain embodiments of the inventive formulation (Forms 26, 27, 28, 29 & 30), Control 1 and Control 2 in male minipigs (Goettingen) and Control 1 formulation in human.

FIG. 12 (A) depicts half-life, Cmax, Tmax and AUC for certain embodiments of the inventive formulation (Forms 26, 27, 28 & 29), Control 1 and Control 2 in male minipigs (Goettingen) and Control 1 formulation; (B) depicts mean (±SEM) plasma concentrations of acetaminophen following oral dose administration of certain embodiments of the inventive formulation (Forms 26, 27, 28 & 29), Control 1 and Control 2 in male minipigs (Goettingen) and Control 1 formulation.

FIG. 13 (A) depicts chemical structure for hydrocodone; (B) depicts half-life, Cmax, Tmax and AUC following oral dose administration of certain embodiments of the inventive formulation (Forms 26, 27, 28 & 29), Control 1 and Control 2 in male minipigs (Goettingen) and Control 1 formulation; (C) depicts mean (±SEM) plasma concentrations of hydrocodone following oral dose administration of certain embodiments of the inventive formulation (Forms 26, 27, 28 & 29), Control 1 and Control 2 in male minipigs (Goettingen) and Control 1 formulation.

FIG. 14 depicts the rate of dissolution of various drug dosage forms 32-37 with respect to hydrocodone in 20% aqueous ethanol.

FIG. 15 depicts the rate of dissolution of various drug dosage forms 32-37 with respect to hydrocodone in 0.01 N hydrochloric acid.

FIG. 16 depicts the rate of dissolution of drug dosage form 31 with respect to hydrocodone in 0.01 N hydrochloric acid directly after manufacturing and after storage for 1 month at 25° C./60% relative humidity, at 40° C./75% relative humidity, and at 60° C. dry, respectively.

FIG. 17 depicts rate of dissolution of drug dosage form 31 with respect to acetaminophen (APAP) in 0.01 N hydrochloric acid directly after manufacturing and after storage for 1 month at 25° C./60% relative humidity, at 40° C./75% relative humidity, and at 60° C. dry, respectively.

FIG. 18 depicts rate of dissolution of various drug dosage forms 32, 34, and 36 with respect to acetaminophen (APAP) in 0.01 N hydrochloric acid+5% NaCl.

FIG. 19 depicts rate of dissolution of various drug dosage forms 32, 34, and 36 with respect to acetaminophen (APAP) in 0.05 M phosphate buffer pH 6.78.

FIG. 20 depicts rate of dissolution of various drug dosage forms 32, 34, and 36 with respect to acetaminophen (APAP) in 0.01 N HCl and 0.09% NaCl.

FIG. 21 depicts rate of dissolution of various drug dosage forms 32, 34, and 36 with respect to acetaminophen (APAP) in 0.01 N HCl.

FIG. 22 depicts rate of dissolution of various drug dosage forms 38-40 with respect to hydrocodone in 0.01 N HCl.

FIG. 23 depicts rate of dissolution of various drug dosage forms 38-40 with respect to acetaminophen (APAP) in 0.01 N HCl.

FIG. 24 depicts rate of dissolution of various drug dosage forms 38-40 with respect to hydrocodone in 40% aqueous ethanol.

FIG. 25 depicts rate of dissolution of various drug dosage forms 38-40 with respect to acetaminophen (APAP) in 40% aqueous ethanol.

FIG. 27 depicts mean acetaminophen concentration-time profiles for Form 45 and Control 1.

FIGS. 28 A and B depicts hydrocodone concentration-time profile for individual subject for Form 45 and Control 1, respectively.

FIGS. 29 A and B depicts acetaminophen concentration-time profile for individual subject for Form 45 and Control 1, respectively.

FIGS. 30 A and B depicts mean hydrocodone concentration-time profile for period 1 and 2, respectively for Form 45 and Control 1.

FIGS. 31 A and B depicts mean acetaminophen concentration-time profile by periods 1 and 2, respectively for Form 45 and Control 1.

FIGS. 32 A and B depicts mean hydrocodone and acetaminophen concentrations for in vitro Form 45, in vitro Control 1, in vivo Control 1 concentration and in vitro-in vivo concentration predictions for Form 45.

FIGS. 33 A and B depicts mean hydrocodone and acetaminophen in vitro dissolution profiles for Form 45 and Control 1

### DETAILED DESCRIPTION OF THE INVENTION

The invention is not limited to the particular methodology, protocols, animal studies, and reagents described, which can vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, animals, instruments, statistical analysis and methodologies which are reported in the publications which might be used in connection with the invention. Nothing herein is to be construed as an admission that the invention is not entitled to antedate such disclosure by virtue of prior invention.

Trademarks are used in this description as a convenient abbreviation for well known materials. As one of ordinary skill would appreciate, the following brand names indicate the substances indicated:

### EUDRAGIT®: Polymers derived from esters of acrylic and methacrylic acid;

### METHOCEL®: Methyl or methoxyl Cellulose

### KOLLICOAT®: Polyvinyl alcohol-polyethylene glycol-graft copolymers

### PLASDONE®: Polyvinylpyrrolidone polymer or -copolymer

### LAUROGLYCOL®: Propylene glycol laurate ester

### SPAN®: Sorbitan fatty acid esters

### CREMOPHOR®: Polyethoxylated Castor oil

### POLOXAMER®: Polyoxyethylene polyoxypropylene block copolymers or polyoxyethylene polypropyleneglycol

### TWEEN®: Polyethoxylated Sorbitan esters

### KLUCEL®: Hydroxypropylcellulose

### KOLLIDON®: Polyvinlypyrrolidone homo- or copolymers

### XYLITOL®: (2,3,4,5)tetrahydroxy-pentanol

### ISOMALT®: An equimolar composition of 6-0-α-D-glucopyranosido-D-sorbitol (1,6-GPS) and 1-O-α-D-glucopyranosido-D-mannitol-dihydrate (1,1-GPM-dihydrate).

### POLYOX®: Water-Soluble Resins based on polyethyleneoxide

### XYLIT®: (2,3,4,5)tetrahydroxy-pentanol

### PLUROL OLEIQUE®: Oleic esters of polyglycerol

### LUTROL®: Polyoxyethylene polyoxypropylene block copolymers or polyoxyethylene olypropyleneglycol

### ETHOCEL®: Ethylcellulose

### PRIMOJEL®: Sodium starch glycolate

The present invention provides an improved solid or solid solution, oral dosage formulation that provides for the in vivo sustained-release of pharmaceutically active compounds (“drugs”) that have properties that make them likely to be abused or have been shown to be frequently abused, as well as salts, esters, prodrugs and other pharmaceutically-acceptable equivalents thereof.

The term “AUC” refers to the area under the concentration time curve, calculated using the trapezoidal rule and Clast/k, where Clast is the last observed concentration and k is the calculated elimination rate constant.

The term “AUCt” refers to the area under the concentration time curve to last observed concentration calculated using the trapezoidal rule.

The term “Cmax” refers to the plasma concentration of the referent abuse relevant drug at Tmax, expressed as ng/mL and μg/mL, respectively, produced by the oral ingestion of a composition of the invention. Unless specifically indicated, Cmax refers to the overall maximum observed concentration.

The term “Cmin” refers to the minimum observed concentration within the intended dosing interval, e.g., a twelve hour dosing interval for a formulation labelled as suitable for dosing every 12 hours or as needed, of a dosage form of the invention administered for 5 doses contiguous dosing intervals.

The term “ng*hr/mL/mg” refers to the amount of the substance measured in nanograms times the number of hours per milliliter of blood divided by the milligrams of the abuse relevant drug administered to the animal or human.

As used herein, the phrase “ascending release rate” refers to a dissolution rate that generally increases over time, such that the drug dissolves in the fluid at the environment of use at a rate that generally increases with time, rather than remaining constant or decreasing, until the dosage form is depleted of about 80% of the drug.

In one preferred embodiment, the invention provides dosage forms that inhibit the extraction of the drug by common solvents, e.g., without limitation, distilled aqueous ethanol, from the formulation. The formulation dissuades abuse by limiting the ability of persons to extract the opioid from the formulation (either intentionally or unintentionally), such that the opioid cannot easily be concentrated for parenteral administration. Also these abuse resistant formulations may not be easily broken down into smaller particulates or powder-form that are easily abused by nasal snorting. Such an abuse-resistant formulation does not require incorporation of an opioid antagonist (albeit, an opioid antagonist may be added to the preparation to further dissuade abuse). While not desiring to be bound by any particular theory, it is believed that incorporation of alkylcelluloses, such as (without limitation) hydroxymethylcelluloses, and preferably hydroxypropylmethylcelluloses contribute to the formulation's resistance to extraction in alcohol, particularly in 20% or 40% aqueous ethanol. The alkylcellulose preferably has at least 12% substitution with an alkylsubstituent, more preferably at least 16% substitution with an alkyl substituent, and most preferably at least 19% substitution with an alkyl substituent. Alkyl substitutions of the cellulose below about 40%, and more preferably below about 30%, are preferred in the context of the invention. Additionally, the alkyl substituent is preferably C₁-C₆, more preferably C₁, C₂ or C₄, and most preferably C₃, and can be straight-chained or branched when the alkyl substituent contains 3 or more carbon atoms.

In another preferred embodiment, the dosage forms optionally resists cutting, grinding, pulverization and the like. A convenient measure for this aspect of the invention is “breaking strength,” as measured by “Pharma Test PTB 501” hardness tester. The inventive formulation preferably has a breaking strength of at least 150 newtons (150 N). More preferably, the inventive formulation has breaking strength of at least 300 N, yet more preferably of at least 450 N, and yet more preferably of at least 600 N.

Breaking strength according to the present invention can be determined with a tablet 10 mm in diameter and 5 mm in width according to the method for determining the breaking strength of tablets published in the European Pharmacopoeia 1997, page 143, 144, method no. 2.9.8. A preferred apparatus used to measure breaking strength is a “Zwick Z 2.5” materials tester, Fmax=2.5 kN, draw max. 1150 mm with the set up comprising a column and a spindle, clearance behind of 100 mm, a n d a test speed of 0.1800 mm/min. Measurement can be performed using a pressure piston with screw-in inserts and a cylinder (10 mm diameter), a force transducer, (Fmax. 1 kN, diameter=8 mm, class 0.5 from 10 N, class I from 2 N to ISO 7500-1, Zwick gross force Fmax=1.45 kN). The apparatus can optionally be obtained from Zwick GmbH & Co. KG, Ulm, Germany.

Any suitable means can be used to produce the inventive composition. In a preferred embodiment, the formulation is preferably melt-processed, and more preferably melt-extruded, and then in either case directly shaped without milling or grinding the formulation. Notwithstanding the foregoing, it is contemplated that the directly shaped tablets of the formulation can be optionally coated with a swallowing aid, such as without limitation, a gelatin coat. While not desiring to be bound by any particular theory, it is believed that direct shaping to prevent undesirable sharp features from forming on the formulation without an intermediate grinding step contributes to the superior breaking strength of the formulation. Additionally, embodiments of the inventive formulation optionally gain additional breaking strength by employing at least two melt-processed polymers. While not ascribing to any particular theory, it is believed that the second melt-processed polymer preferentially interacts with the first melt-processed polymer so as to advantageously adjust the transition glass temperature of the composition as a whole during the formation of the tablet.

In one embodiment, the formulation may use a polymer, or a copolymer, or a combination thereof to create the melt-processed, and more preferably melt-extruded, directly shaped formulation. Polymers that are pharmacologically inactive and provide enteric coatings or sustained release profile for the formulation can also be used. In one embodiment, suitable polymers/copolymers include poly(meth)acrylate like e.g. Eudragit L- or S-type, which are pharmacologically inactive.

EUDRAGIT® is a tradename for some preferred polymers that are suitable for use in the invention and are derived from esters of acrylic and methacrylic acid. The properties of the EUDRAGIT polymers are principally determined by functional groups incorporated into the monomers of the EUDRAGIT polymers. The individual EUDRAGIT® grades differ in their proportion of neutral, alkaline or acid groups and thus in terms of physicochemical properties. Ammonioalklyl methacrylate copolymers or methacrylate copolymers may be used having the following formula:

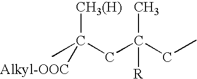

### According to 2007 US Pharmacopoeia Eudragit is defined according to USP 30/NF 25

### Methacrylic acid copolymer, type A N F=Eudragit L-100

### Methacrylic acid copolymer, type B N F=Eudragit S-100

### Methacrylic acid copolymer, type C NF=Eudragit L-100-55 (contains a small detergent amount)

### Ammonio Methacrylate Copolymer, type A NF=Eudragit RL-100 (granules)

### Ammonio Methacrylate Copolymer, type A NF=Eudragit RL-PO (powder)

### Ammonio Methacrylate Copolymer, type B NF=Eudragit RS-100 (granules)

### Ammonio Methacrylate Copolymer, type B NF=Eudragit RS-PO (powder)

Polyacrylate Dispersion 30 Percent Ph. Eur.=Eudragit NE30D (=30% aqueous dispersion)
Basic butylated methacrylate copolymer Ph. Eur.=Eudragit E-100

wherein the functional group has a quaternary ammonium (trimethylammonioethyl methacrylate) moiety or R=COOCH₂CH₂N⁺(CH₃)₃Cl⁻ [commercially available as EUDRAGIT® (RL or RS)] or the functional group is a carboxylic acid, or R=COOH [commercially available as EUDRAGIT® (L)]. When the functional group is a carboxylic acid moiety, the EUDRAGIT® (L) polymer is gastroresistant and enterosoluble. Thus formulations using EUDRAGIT® (L) will be resistant to gastric fluid and will release the active agent in the colon. When the functional group is a trimethylammonioethyl methacrylate moiety, the EUDRAGIT® (RL or RS) polymers are insoluble, permeable, dispersible and pH-independent. These EUDRAGIT® (RL or RS) polymers may therefore be used for delayed drug release for sustained release formulations. EUDRAGIT® is sold in various forms such as in solid form (EUDRAGIT® L100/S100/L-100-55, EUDRAGIT® E PO, EUDRAGIT® RL PO, Eudragit RS PO), granules (EUDRAGIT® E100, EUDRAGIT®RL 100/RS 100), dispersions (L 30 D-55/FS 30D 30%, EUDRAGIT® NE 30 D/40 D 30%/40% polymer content, EUDRAGIT®RL 30 D RS 30 D 30%) and organic solutions (EUDRAGIT® L 12.5, EUDRAGIT® E12.5, EUDRAGIT® RL 12.5/RS 12.5-12.5% organic solution).

When at least two melt-processed polymers are employed, one is preferably a cellulose derivative, more preferably a hydroxyalkylcellulose derivative, and optionally hydroxypropylmethylcellulose, and independently, the other polymer is preferably a (meth)acrylate polymer (such as, any suitable Eudragit polymer). Among the (meth)acrylate polymer polymers preferred in the context of the invention are Eudragit L and Eudragit RS. One more preferred polymer in the context of the invention is Eudragit RL. The Eudragit polymers can be used in combinations, with mixtures of Eudragit RS and RL being preferred.

Persons that (albeit inadvisedly) drink substantial quantities of alcoholic beverages when taking physician prescribed medications can substantially alter the composition of the gastric juices contained in the stomach, and in extreme cases these gastric juices can comprise up to 40% alcohol. Advantageously, embodiments of the inventive abuse-deterrent formulation optionally comprises a melt-processed mixture of at least one abuse-relevant drug, at least one cellulose ether or cellulose ester, and at least one (meth)acrylic polymer, wherein the amount of the drug that is extracted from the formulation by 20% aqueous ethanol, or 40% aqueous ethanol, or both within one hour at 37° C. is less than or equal twice the amount of the drug that is extracted by 0.01 N hydrochloric acid within one hour at 37° C., or at 25° C. or both. The resistance to extraction by 40% ethanol is advantageous in those situations in which an individual purposefully attempts to extract an abuse relevant drug from a medicine containing an abuse relevant drug.

The protocols for extraction by 20% or 40% aqueous ethanol or 0.01 N hydrochloric acid, respectively, are given in the experimental section that follows. In more preferred embodiments, the amount of the drug that is extracted from the formulation by 20% or 40% aqueous ethanol is less than or equal 1.5 times the amount of the drug that is extracted by 0.01 N hydrochloric acid within one hour. In a yet more preferred embodiments, the amount of the drug that is extracted from the formulation by 20% or 40% aqueous ethanol is less than or equal the amount of the drug that is extracted by 0.01 N hydrochloric acid within one hour. In a yet more preferred embodiments, the amount of the drug that is extracted from the formulation by 20% or 40% aqueous ethanol is less than or equal 0.9 times the amount of the drug that is extracted by 0.01 N hydrochloric acid within one hour.

The present invention also provides a sustained release formulation of at least one abuse relevant drug that hampers the extraction of the drug from the formulation when extraction is by solvent extraction with commonly available household extraction solvents such as isopropyl alcohol, distilled alcohols exemplified by vodka, white vinegar, water and aqueous ethanol (e.g., 20% ethanol). Whereas the formulation is largely resistant to solvent-extraction, it still provides adequate drug release in aqueous solutions such as gastric fluids. This formulation when crushed or ground also provides adequate drug release in aqueous solutions such as gastric fluids. Fortunately, in certain preferred embodiments of the invention, the amount of the abuse relevant drug released from the time of placing in 3 oz. of one, or two, or three, or more than three, of the household solvents listed above (i.e., 0 hours) to 1 hour is not more than 15% greater than the amount released over the same time as when swallowed by an ordinary human, or the more than 1 hour to about 4 hours is not more than 15% greater than the amount released over the same time as when swallowed by an ordinary human, or both.

Exemplary preferred compositions of the invention comprise:

Cellulose ethers and cellulose esters, which can be used alone or in combination in the invention have a preferable molecular weight in the range of 50,000 to 1,250,000 daltons. Cellulose ethers are preferably selected from alkylcelluloses, hydroxalkylcelluloses, hydroxyalkyl alkylcelluloses or mixtures therefrom, such as ethylcellulose, methylcellulose, hydroxypropyl cellulose (NF), hydroxyethyl cellulose (NF), and hydroxpropyl methylcellulose (USP), or combinations thereof. Useful cellulose esters are, without limitation, cellulose acetate (NF), cellulose acetate butyrate, cellulose acetate propionate, hydroxypropylmethyl cellulose phthalate, hydroxypropylmethyl cellulose acetate phthalate, and mixtures thereof. Most preferably, non-ionic polymers, such as hydroxypropylmethyl cellulose may be used.

The amount of substituent groups on the anhydroglucose units of cellulose can be designated by the average number of substituent groups attached to the ring, a concept known to cellulose chemists as “degree of substitution” (D. S.). If all three available positions on each unit are substituted, the D. S. is designated as 3, if an average of two on each ring are reacted, the D. S. is designated as 2, etc.

In preferred embodiments, the cellulose ether has an alkyl degree of substitution of 1.3 to 2.0 and hydroxyalkyl molar substitution of up to 0.85.

In preferred embodiments, the alkyl substitution is methyl. Further, the preferred hydroxyalkyl substitution is hydroxpropyl. These types of polymers with different substitution degrees of methoxy- and hydroxypropoxy-substitutions are summarized listed in pharmacopoeas, e.g. USP under the name “Hypromellose”.

Methylcellulose is available under the brand name METHOCEL A. METHOCEL A has a methyl (or methoxyl) D. S. of 1.64 to 1.92. These types of polymers are listed in pharmacopoeas, e.g. USP under the name “Methylcellulose”.

A particularly preferred cellulose ether is hydroxpropyl methylcellulose. Hydroxpropyl methylcellulose is available under the brand name METHOCEL E (methyl D. S. about 1.9, hydroxypropyl molar substitution about 0.23), METHOCEL F (methyl D. S. about 1.8, hydroxypropyl molar substitution about 0.13), and METHOCEL K (methyl D. S. about 1.4, hydroxypropyl molar substitution about 0.21). METHOCEL F and METHOCEL K are preferred hydroxpropyl methylcelluloses for use in the present invention.

The acrylic polymer suitably includes homopolymers and copolymers (which term includes polymers having more than two different repeat units) comprising monomers of acrylic acid and/or alkacrylic acid and/or an alkyl (alk)acrylate. As used herein, the term “alkyl (alk)acrylate” refers to either the corresponding acrylate or alkacrylate ester, which are usually formed from the corresponding acrylic or alkacrylic acids, respectively. In other words, the term “alkyl (alk)acrylate” refers to either an alkyl alkacrylate or an alkyl acrylate.

Preferably, the alkyl (alk)acrylate is a (C₁-C₂₂)alkyl ((C₁-C₁₀)alk)acrylate. Examples of C₁-C₂₂ alkyl groups of the alkyl (alk)acrylates include methyl, ethyl, n-propyl, n-butyl, iso-butyl, tert-butyl, iso-propyl, pentyl, hexyl, cyclohexyl, 2-ethyl hexyl, heptyl, octyl, nonyl, decyl, isodecyl, undecyl, dodecyl, tridecyl, tetradecyl, pentadecyl, hexadecyl, heptadecyl, octadecyl, nonadecyl, eicosyl, behenyl, and isomers thereof. The alkyl group may be straight or branched chain. Preferably, the (C₁-C₂₂)alkyl group represents a (C₁-C₆)alkyl group as defined above, more preferably a (C₁-C₄)alkyl group as defined above. Examples of C₁₋₁₀ alk groups of the alkyl (alk)acrylate include methyl, ethyl, n-propyl, iso-propyl, n-butyl, iso-butyl, tert-butyl, pentyl, hexyl, cyclohexyl, 2-ethyl hexyl, heptyl, octyl, nonyl, decyl and isomers thereof. The alk groups may be straight or branched chain. Preferably, the (C₁-C₁₀)alk group represents a (C₁-C₆)alk group as defined above, more preferably a (C₁-C₄) alk group as defined above.

Preferably, the alkyl (alk)acrylate is a (C₁-C₄)alkyl ((C₁-C₄) alk)acrylate, most preferably a (C₁-C₄)alkyl (meth)acrylate. It will be appreciated that the term (C₁-C₄)alkyl (meth)acrylate refers to either (C₁-C₄)alkyl acrylate or (C₁-C₄)alkyl methacrylate. Examples of (C₁-C₄)alkyl (meth)acrylate include methyl methacrylate (MMA), ethyl methacrylate (EMA), n-propyl methacrylate (PMA), isopropyl methacrylate (IPMA), n-butyl methacrylate (BMA), isobutyl methacrylate (IBMA), tert-butyl methacrylate (TBMA): methyl acrylate (MA), ethyl acrylate (EA), n-propyl acrylate (PA), n-butyl acrylate (BA), isopropyl acrylate (IPA), isobutyl acrylate (IBA), and combinations thereof.

Preferably, the alkacrylic acid monomer is a (C₁-C₁₀)alkacrylic acid. Examples of (C₁-C₁₀)alkacrylic acids include methacrylic acid, ethacrylic acid, n-propacrylic acid, iso-propacrylic acid, n-butacrylic acid, iso-butacrylic acid, tert-butacrylic acid, pentacrylic acid, hexacrylic acid, heptacrylic acid and isomers thereof. Preferably the (C₁-C₁₀)alkacrylic acid is a (C₁-C₄)alkacrylic acid, most preferably methacrylic acid.

In certain embodiments, the alkyl groups may be substituted by aryl groups. As used herein “alkyl” group refers to a straight chain, branched or cyclic, saturated or unsaturated aliphatic hydrocarbons. The alkyl group has 1-16 carbons, and may be unsubstituted or substituted by one or more groups selected from halogen, hydroxy, alkoxy carbonyl, amido, alkylamido, dialkylamido, nitro, amino, alkylamino, dialkylamino, carboxyl, thio and thioalkyl. A “hydroxy” group refers to an OH group. An “alkoxy” group refers to an —O-alkyl group wherein alkyl is as defined above. A “thio” group refers to an —SH group. A “thioalkyl” group refers to an —SR group wherein R is alkyl as defined above. An “amino” group refers to an —NH₂ group. An “alkylamino” group refers to an —NHR group wherein R is alkyl is as defined above. A “dialkylamino” group refers to an —NRR′ group wherein R and R′ are all as defined above. An “amido” group refers to an —CONH₂. An “alkylamido” group refers to an —CONHR group wherein R is alkyl is as defined above. A “dialkylamido” group refers to an —CONRR′ group wherein R and R′ are alkyl as defined above. A “nitro” group refers to an NO₂ group. A “carboxyl” group refers to a COOH group.

In certain embodiments, the alkyl groups may be substituted by aryl groups. As used herein, “aryl” includes both carbocyclic and heterocyclic aromatic rings, both monocyclic and fused polycyclic, where the aromatic rings can be 5- or 6-membered rings. Representative monocyclic aryl groups include, but are not limited to, phenyl, furanyl, pyrrolyl, thienyl, pyridinyl, pyrimidinyl, oxazolyl, isoxazolyl, pyrazolyl, imidazolyl, thiazolyl, isothiazolyl and the like. Fused polycyclic aryl groups are those aromatic groups that include a 5- or 6-membered aromatic or heteroaromatic ring as one or more rings in a fused ring system. Representative fused polycyclic aryl groups include naphthalene, anthracene, indolizine, indole, isoindole, benzofuran, benzothiophene, indazole, benzimidazole, benzthiazole, purine, quinoline, isoquinoline, cinnoline, phthalazine, quinazoline, quinoxaline, 1,8-naphthyridine, pteridine, carbazole, acridine, phenazine, phenothiazine, phenoxazine, and azulene. Also as used herein, aryl group also includes an arylalkyl group. Further, as used herein “arylalkyl” refers to moieties, such as benzyl, wherein an aromatic is linked to an alkyl group.

Preferably, the acrylic polymer is an acrylic copolymer. Preferably, the acrylic copolymer comprises monomers derived from alkyl (alk)acrylate, and/or acrylic acid and/or alkacrylic acid as defined hereinbefore. Most preferably, the acrylic copolymer comprises monomers derived from alkyl (alk)acrylate, i.e. copolymerisable alkyl acrylate and alkyl alkacrylate monomers as defined hereinbefore. Especially preferred acrylic copolymers include a (C₁-C₄)alkyl acrylate monomer and a copolymerisable (C₁-C₄)alkyl (C₁-C₄)alkacrylate comonomer, particularly copolymers formed from methyl methacrylate and a copolymerisable comonomer of methyl acrylate and/or ethyl acrylate and/or n-butyl acrylate.

Preferably, the (meth)acrylic polymer is a ionic (meth)acrylic polymer, in particular a cationic (meth)acrylic polymer. Ionic (meth)acrylic polymer are manufactured by copolymerising (meth)acrylic monomers carrying ionic groups with neutral (meth)acrylic monomers. The ionic groups preferably are quaternary ammonium groups.

The (meth)acrylic polymers are generally water-insoluble, but are swellable and permeable in aqueous solutions and digestive fluids. The molar ratio of cationic groups to the neutral (meth)acrylic esters allows for are control of the water-permeability of the formulation. In preferred embodiments the (meth)acrylic polymer is a copolymer or mixture of copolymers wherein the molar ratio of cationic groups to the neutral (meth)acrylic esters is in the range of about 1:20 to 1:35 on average. The ratio can by adjusted by selecting an appropriate commercially available cationic (meth)acrylic polymer or by blending a cationic (meth)acrylic polymer with a suitable amount of a neutral (meth)acrylic polymer.

Suitable (meth)acrylic polymers are commercially available from Rohm Pharma under the Tradename Eudragit, preferably Eudragit RL and Eudragit RS. Eudragit RL and Eudragit RS are copolymers of acrylic and methacrylic esters with a low content of quaternary ammonium groups, the molar ratio of ammonium groups to the remaining neutral (meth)acrylic esters being 1:20 in Eudragit RL and 1:40 in Eudragit RS. The mean molecular weight is about 150,000.

Besides the (meth)acrylic polymers, further pharmaceutically acceptable polymers may be incorporated in the inventive formulations in order to adjust the properties of the formulation and/or improve the ease of manufacture thereof. These polymers may be selected from the group comprising:

homopolymers of N-vinyl lactams, especially polyvinylpyrrolidone (PVP),

copolymers of a N-vinyl lactam and one or more comonomers copolymerizable therewith, the comonomers being selected from nitrogen-containing monomers and oxygen-containing monomers; especially a copolymer of N-vinyl pyrrolidone and a vinyl carboxylate, preferred examples being a copolymer of N-vinyl pyrrolidone and vinyl acetate or a copolymer of N-vinyl pyrrolidone and vinyl propionate;

polyvinyl alcohol-polyethylene glycol-graft copolymers (available as, e.g., Kollicoat® IR from BASF AG, Ludwigshafen, Germany);

high molecular polyalkylene oxides such as polyethylene oxide and polypropylene oxide and copolymers of ethylene oxide and propylene oxide;

polyacrylamides;

vinyl acetate polymers such as copolymers of vinyl acetate and crotonic acid, partially hydrolyzed polyvinyl acetate (also referred to as partially saponified “polyvinyl alcohol”);

polyvinyl alcohol;

poly(hydroxy acids) such as poly(lactic acid), poly(glycolic acid), poly(3-hydroxybutyrate) and poly(3-hydroxybutyrate-co-3-hydroxyvalerate); or mixtures of one or more thereof.

“Abuse-relevant drug” is intended to mean any biologically effective ingredient the distribution of which is subject to regulatory restrictions. Drugs of abuse that can be usefully formulated in the context of the invention include without limitation pseudoephedrine, anti-depressants, strong stimulants, diet drugs, steroids, and non-steroidal anti-inflammatory agents. In the category of strong stimulants, methamphetamine is one drug that has recently received popular attention as a drug of abuse. There is also some concern at the present time about the abuse potential of atropine, hyoscyamine, phenobarbital, scopolamine, and the like. Another major class of abuse-relevant drugs are analgesics, especially the opioids.

By the term “opioid,” it is meant a substance, whether agonist, antagonist, or mixed agonist-antagonist, which reacts with one or more receptor sites bound by endogenous opioid peptides such as the enkephalins, endorphins and the dynorphins. Opioids include, without limitation, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, pentazocine, phenadoxone, phenazocine, phenomorphan, phenoperidine, piminodine, propiram, propoxyphene, sufentanil, tilidine, and tramadol, and salts and mixtures thereof.

In some preferred embodiments, the inventive formulation includes at least one additional therapeutic drug. In even more preferred embodiments, the additional therapeutic dug can be, without limitation, selected from the group consisting of non-steroidal, non-opioidal analgesics, and is optionally further selected from the group consisting of acetaminophen, aspirin, fentaynl, ibuprofen, indomethacin, ketorolac, naproxen, phenacetin, piroxicam, sufentanyl, sunlindac, and interferon alpha. Particularly preferred are those combinations of drug currently sold as fixed dose combinations to the public under the authority of a suitable national or regional regulatory agency, such as (by way of example) the U.S. Food and Drug Administration. Such drugs include without limitation a (fixed dose) combination of hydrocodone and acetaminophen, or a (fixed dose) combination of hydrocodone and ibuprofen.

The abuse-relevant drug(s) are preferably dispersed evenly throughout a matrix that is preferably formed by a cellulose ether or cellulose ester, and one acrylic or methacrylic polymer as well as other optional ingredients of the formulation. This description is intended to also encompass systems having small particles, typically of less than 1 μm in diameter, of drug in the matrix phase. These systems preferably do not contain significant amounts of active opioid ingredients in their crystalline or microcrystalline state, as evidenced by thermal analysis (DSC) or X-ray diffraction analysis (WAXS). At least 98% (by weight) of the total amount of drug is preferably present in an amorphous state. If additional non-abuse relevant drug actives like e.g. acetaminophen are additionally present in a formulation according to the present invention, this additional drug active(s) may be in a crystalline state embedded in the formulation.

When the dispersion of the components is such that the system is chemically and physically uniform or substantially homogenous throughout or consists of one thermodynamic phase, such a dispersion is called a “solid solution”. Solid solutions of abuse-relevant actives are preferred.

The formulation can also comprise one or more additives selected from sugar alcohols or derivatives thereof, maltodextrines; pharmaceutically acceptable surfactants, flow regulators, disintegrants, bulking agents and lubricants. Useful sugar alcohols are exemplified by mannitol, sorbitol, xylitol; useful sugar alcohol derivatives include without limitation isomalt, hydrogenated condensed palatinose and others that are both similar and dissimilar.

Pharmaceutically acceptable surfactants are preferably pharmaceutically acceptable non-ionic surfactant. Incorporation of surfactants is especially preferred for matrices containing poorly water-soluble active ingredients and/or to improve the wettability of the formulation. The surfactant can effectuate an instantaneous emulsification of the active ingredient released from the dosage form and prevent precipitation of the active ingredient in the aqueous fluids of the gastrointestinal tract.

Some preferred additives include polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; polyoxyethylene alkylaryl ethers, e.g. polyoxyethylene (2) nonylphenyl ether, polyoxyethylene (3) nonylphenyl ether, polyoxyethylene (4) nonylphenyl ether or polyoxyethylene (3) octylphenyl ether; polyethylene glycol fatty acid esters, e.g. PEG-200 monolaurate, PEG-200 dilaurate, PEG-300 dilaurate, PEG-400 dilaurate, PEG-300 distearate or PEG-300 dioleate; alkylene glycol fatty acid mono esters, e.g. propylene glycol mono- and dilaurate (Lauroglycol®); sucrose fatty acid esters, e.g. sucrose monostearate, sucrose distearate, sucrose monolaurate or sucrose dilaurate; sorbitan fatty acid mono- and diesters such as sorbitan mono laurate (Span® 20), sorbitan monooleate, sorbitan monopalmitate (Span® 40), or sorbitan stearate, polyoxyethylene castor oil derivates, e.g. polyoxyethyleneglycerol triricinoleate or polyoxyl 35 castor oil (Cremophor® EL; BASF Corp.) or polyoxyethyleneglycerol oxystearate such as polyethylenglycol 40 hydrogenated castor oil (Cremophor® RH 40) or polyethylenglycol 60 hydrogenated castor oil (Cremophor® RH 60); or block copolymers of ethylene oxide and propylene oxide, also known as polyoxyethylene polyoxypropylene block copolymers or polyoxyethylene polypropyleneglycol such as Pluronic® F68, Pluronic® F127, Poloxamer® 124, Poloxamer® 188, Poloxamer® 237, Poloxamer® 388, or Poloxamer® 407 (BASF Wyandotte Corp.); or mono fatty acid esters of polyoxyethylene (20) sorbitan, e.g. polyoxyethylene (20) sorbitan monooleate (Tween® 80), polyoxyethylene (20) sorbitan monostearate (Tween® 60), polyoxyethylene (20) sorbitan monopalmitate (Tween® 40), polyoxyethylene (20) sorbitan monolaurate (Tween® 20), and the like as well as mixtures of two, three, four, five, or more thereof.

Various other additives may be included in the melt, for example flow regulators such as colloidal silica; lubricants, fillers, disintegrants, plasticizers, stabilizers such as antioxidants, light stabilizers, radical scavengers or stabilizers against microbial attack.

The formulations of the invention can be obtained through any suitable melt process such as by the use of a heated press, and are preferably prepared by melt extrusion. In order to obtain a homogeneous distribution and a sufficient degree of dispersion of the drug, the drug-containing melt can be kept in the heated barrel of a melt extruder during a sufficient residence time. Melting occurs at the transition into a liquid or rubbery state in which it is possible for one component to be homogeneously embedded in the other. Melting usually involves heating above the softening point of a cellulose ether/ester or (meth)acrylic polymer. The preparation of the melt can take place in a variety of ways.

Usually, the melt temperature is in the range of 70 to 250° C., preferably 80 to 180° C., most preferably 100 to 140° C.

When the melt process comprises melt extrusion, the melting and/or mixing can take place in an apparatus customarily used for this purpose. Particularly suitable are extruders or kneaders. Suitable extruders include single screw extruders, intermeshing screw extruders, and multiscrew extruders, preferably twin screw extruders, which can be co-rotating or counter rotating and are optionally equipped with kneading disks. It will be appreciated that the working temperatures will also be determined by the kind of extruder or the kind of configuration within the extruder that is used. Part of the energy needed to melt, mix and dissolve the components in the extruder can be provided by heating elements. However, the friction and shearing of the material in the extruder may also provide the mixture with a substantial amount of energy and aid in the formation of a homogeneous melt of the components.

In another embodiment, the invention provides an oral, sustained release dosage form characterized in that it has at least two of the following features (a) the drug that is extracted from the formulation by ethanolic solvent, e.g. 40% or 20% aqueous ethanol or both within one hour at 37° C., with or without agitation, is less than or equal twice the amount of the drug that is extracted by 0.01 N hydrochloric acid within one hour at 37° C., (b) the dosage form is resistant to tampering and does not break under a force of 300 newtons, preferably 600 newtons, more preferably 1200 newtons, as measured by “Pharma Test PTB 501” hardness tester, and (c) the dosage form releases at least 15%, more preferably 18%, and optionally 24% of the drug, but not more than 45%, more preferably 38% and optionally 34% of the drug during the 30 minute, first hour, or first two hours in in vitro dissolution testing and optionally also in vivo (i.e., in the digestive tract of an animal or human). While not desiring to be bound by any particular theory, it is believed that high initial release rate of drug from the formulation are accomplished by providing a high drug load in the formulation. Drug loading for a single active ingredient, such as acetaminophen in some embodiments of the inventive formulation can be greater than about 60%, 70%, 75%, 80%, 85%, by weight. The drug loading of acetaminophen can be limited to 80%.

A preferred embodiment of this dosage form is a monolithic form or a solid solution. The term “monolithic” is derived from roots meaning “single” and “stone”. A monolithic form or a solid preferably has at least one dimension that is more than 5 mm. In monolithic embodiments of the invention, the abuse relevant drug is preferably contained in a single solid, or a single solid solution, element. The monolithic solid or solid solution can optionally be overcoated or combined with other materials. These other materials preferably do not contain a substantial amount of the abuse relevant drug and these materials preferably do not substantially affect the rate of dissolution or dispersion of the abuse relevant drug in vivo or in vitro. The in vitro and/or in vivo release rates of the abuse relevant drug or abuse relevant drugs after about the first hour are preferably substantially constant for at least about 6, 8, 10, 12, or 16 hours. Thus, embodiments of the invention provides a single phase drug formulation that can be adapted to provide a burst of the abuse relevant drug(s) to allow therapeutic levels of the drug to be quickly obtained in the blood of a patient or animal, and to be maintained to provide therapeutic quantities for at least about 8, 12, or 24 hours. Additionally, the drug formulation is preferably suitable for repeated administration to a human or animal once, twice or three times a day.

Advantageously, preferred embodiments of the inventive dosage form release substantially the entire quantity of the abuse relevant drug incorporated into the dosage form. For example, the inventive dosage form can be adapted to deliver greater than 90%, and preferably 95%, of the drug in in vitro dissolution testing within about 16, and optionally 12 or 9 hours. The cumulative blood concentration, or AUC, cannot be directly known from the time at which 90% of the drug is released from the formulation, however, in general higher AUCs per mg of the abuse relevant drug can be achieved when the drug formulation releases substantially all, or all, of the abuse relevant drug in portions of the digestive tract capable of absorbing the drug into the patient's (or animals) blood system.

In yet another preferred embodiment the invention provides a process for the manufacture of an abuse-resistant drug dosage formulation comprising melt extruding a formulation comprising at least one therapeutic drug further comprising directly shaping the extrudate into a dosage form without (an intermediate) milling step. The melt-extrudate preferably comprises a cellulose derivative, and preferably also comprises a Eudragit polymer. Preferred Eudragit polymers include Eudragit L or Eudragit RS or both, and particularly preferred is Eudragit RL or a combination of Eudragit RL and Eudragit RS.

The melt can range from pasty to viscous. Before allowing the melt to solidify, the melt optionally can be shaped into virtually any desired shape. Conveniently, shaping of the extrudate optionally can be carried out by a calender, preferably with two counter-rotating rollers with mutually matching depressions on their surface. A broad range of tablet forms can be obtained by using rollers with different forms of depressions. Alternatively, the extrudate can be cut into pieces, either before (“hot-cut”) or after solidification (“cold-cut”) or used in a die injection process. Melt processes involving heated presses optionally can also be calendered.

The formed melt can be optionally overcoated with materials that do not contain substantial amount of the drug with abuse potential. For example, the monolithic dosage form containing the drug of abuse can be overcoated with a color coat, a swallowing aid, or another layer of pharmaceutically acceptable materials. The materials layered over the monolithic form preferably do not materially alter the rate of release of the active ingredient from the dosage form.

In order to facilitate the intake of such a dosage form by a mammal, it is advantageous to give the dosage form an appropriate shape. Large tablets that can be swallowed comfortably are therefore preferably elongated rather than round in shape.

A film coat on the dosage form further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropylcellulose, and acrylate or methacrylate copolymers. Besides a film-forming polymer, the film-coat may further comprise a plasticizer, e.g. polyethylene glycol, a surfactant, e.g. a Tween® type, and optionally a pigment, e.g., titanium dioxide or iron oxides. The film-coating may also comprise talc as an anti-adhesive. The film coat usually accounts for less than about 5% by weight of the dosage form.

In one embodiment, the present invention provides an abuse-deterrent drug formulation comprising a melt-processed mixture of a) at least one abuse-relevant drug, b) at least one cellulose ether or cellulose ester, and c) at least one alkyl alkacrylate polymer, alkacrylate polymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily.

Preferably, in this embodiment, the cellulose ether has an alkyl degree of substitution of 1.3 to 2.0 and hydroxyalkyl molar substitution of up to 0.85. More preferably, the alkyl substitution is methyl. Most preferably, the hydroxyalkyl substitution is hydroxpropyl. In another aspect of this embodiment, preferably, the cellulose ether is hydroxpropyl methylcellulose.

In yet another aspect of this embodiment, the alkyl alkacrylate or the alkacrylate polymer has monomeric units of (C₁-C₂₂)alkyl ((C₁-C₁₀)alk)acrylate or (C₁-C₁₀)alkacrylate. More preferably, the alkacrylate polymer is an acrylic polymer or a methacrylic polymer. Also more preferably, the alkacrylate polymer is ionic acrylic polymer or ionic methacrylic polymer. Yet, more preferably, the alkacrylate polymer is a cationic acrylic polymer or cationic methacrylic polymer. Most preferably, the alkacrylate polymer is a copolymer of the acrylic polymer and the methacrylic polymer esters containing quaternary ammonium groups. In the most preferred embodiment, the alkacrylate polymer is a copolymer or mixture of copolymers wherein the molar ratio of cationic groups to the neutral esters is in the range of about 1:20 to 1:35 on average.

In one aspect of this embodiment, the abuse-relevant drug is selected from the group consisting of atropine, hyoscyamine, phenobarbital, and scopolamine salts, esters, prodrugs and mixtures thereof. In another aspect, the abuse-relevant drug is an analgesic, and yet in another aspect, the abuse-relevant drug is an opioid. The opioid may be selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, pentazocine, phenadoxone, phenazocine, phenomorphan, phenoperidine, piminodine, propiram, propoxyphene, sufentanil, tilidine, and tramadol, and salts, esters, prodrugs and mixtures thereof. In another aspect the abuse-relevant drug is selected from the group consisting of pseudoephedrine, anti-depressants, strong stimulants, diet drugs, and non-steroidal anti-inflammatory agents, salts, esters, prodrugs and mixtures thereof. Preferably, the strong stimulant is methamphetamine or amphetamine. The above referenced formulations, also further comprise at least one further drug. In one aspect, further therapeutic drug is selected from the group consisting of non-steroidal, non-opioidal analgesics, and is optionally further selected from the group consisting of acetaminophen, aspirin, fentaynl, ibuprofen, indomethacin, ketorolac, naproxen, phenacetin, piroxicam, sufentanyl, sunlindac, and interferon alpha.

In these formulations, the abuse-relevant drug is preferably dispersed in the formulation in a state of a solid solution. In one aspect, all these formulations may additionally comprise at least one additive independently selected from the group consisting of surfactants, flow regulators, disintegrants, bulking agents, lubricants, effervescent agents, colorants, flavourings, and combinations thereof.

In one embodiment of the invention, between 11% and 47% of the abuse-relevant drug is released in 0.01 N hydrochloric acid within two hours at 37° C. In another embodiment, less than 20% of the abuse-relevant drug is released in 40% aqueous ethanol within one hour at 37° C.

In another embodiment, the present invention provides a monolithic, sustained release oral dosage formulation. This drug formulation comprises a melt-processed mixture of: a) an analgesically effective amount of at least one an abuse-relevant drug, b) at least one cellulose ether or cellulose ester, and c) at least one alkyl alkacrylate polymer, alkacrylate polymer, or a combination thereof. In this formulation, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted for sustained release so as to be useful for oral administration to a human 3, 2, or 1 times daily. Further, in this embodiment, preferably, the cellulose ether has an alkyl degree of substitution of 1.3 to 2.0 and hydroxyalkyl molar substitution of up to 0.85. In another aspect, the alkyl substitution is methyl. In another aspect, the hydroxyalkyl substitution is hydroxpropyl. Preferably, the cellulose ether is hydroxpropyl methylcellulose.

In another aspect of this embodiment, the alkacrylate polymer is an acrylic polymer or a methacrylic polymer. Preferably, the alkacrylate polymer is an ionic acrylic polymer or an ionic methacrylic polymer. More preferably, alkacrylate polymer is a cationic acrylic polymer or a cationic methacrylic polymer. Most preferably, the alkacrylate polymer is a copolymer of the acrylic polymer and the methacrylic polymer esters containing quaternary ammonium groups. Also, more preferably, the acrylic polymer or the methacrylic polymer is a copolymer or mixture of copolymers wherein the molar ratio of cationic groups to the neutral esters is in the range of about 1:20 to 1:35 on average.

In another aspect of this embodiment, the abuse-relevant drug is selected from the group consisting of atropine, hyoscyamine, phenobarbital, and scopolamine salts, esters, prodrugs and mixtures thereof. Preferably, the abuse-relevant drug is an analgesic. More preferably, the abuse-relevant drug is an opioid. Most preferably, the opioid is hydrocodone, its salts and esters. As also described above, the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, pentazocine, phenadoxone, phenazocine, phenomorphan, phenoperidine, piminodine, propiram, propoxyphene, sufentanil, tilidine, and tramadol, and salts, esters, prodrugs and mixtures thereof. Further, the abuse-relevant drug is selected from the group consisting of pseudoephedrine, anti-depressants, strong stimulants, diet drugs, and non-steroidal anti-inflammatory agents, salts, esters, prodrugs and mixtures thereof. Preferably, the strong stimulant is methamphetamine or amphetamine. Another embodiment of the formulation provides at least one further drug. In this embodiment, the further therapeutic drug is selected from the group consisting of non-steroidal, non-opioidal analgesics, and is optionally further selected from the group consisting of acetaminophen, aspirin, fentaynl, ibuprofen, indomethacin, ketorolac, naproxen, phenacetin, piroxicam, sufentanyl, sunlindac, and interferon alpha. Preferably, the abuse-relevant drug is dispersed in the formulation in a state of a solid solution. In another embodiment, the formulation additionally comprises at least one additive selected from the group consisting of surfactants, flow regulators, disintegrants, bulking agents, lubricants, effervescent agents, colorants, flavourings. In one aspect of this embodiment, between 11% and 47% of the abuse-relevant drug is released in 0.01 N hydrochloric acid within two hours at 37° C. In another aspect the dosage form also provides a formulation where less than 20% of the abuse-relevant drug is released in 40% aqueous ethanol within one hour at 37° C.

Another embodiment of the present invention provides an oral sustained release dosage formulation of a drug characterized by at least two of the following features: a) the drug that is extracted from the formulation by 40% aqueous ethanol within one hour at 37° C. is less than or equal twice the amount of the drug that is extracted by 0.01 N hydrochloric acid within one hour at 37° C., b) the formulation does not break under a force of 150 newtons, preferably 300 newtons, more preferably 450 newtons, yet more preferably 500 newtons as measured by “Pharma Test PTB 501” hardness tester, and c) the formulation releases at least 15% of the one drug and not more than 45% of the one drug during the first hour in in vitro dissolution testing and preferably also in vivo. Preferably, in this embodiment, the formulation is not snortable via nasal administration, meaning that when processed in a coffee grinder (as defined hereinabove) for 60 seconds, the material is either uncomfortable for snorting, does not release the abuse relevant drug more than 40 percentage points faster, more preferably less than about 30 percentage points faster, and yet more preferably less than about 20 percentage points faster, than when swallowed with water or with 20% aqueous ethanol or with 40% aqueous ethanol, or both. Also preferably, the drug is an opioid, amphetamine or methamphetamine. More preferably, the formulation comprises an abuse-deterrent drug formulation produced by a melt-processed mixture of a) at least one abuse-relevant drug, b) at least one cellulose ether or cellulose ester, and c) at least one alkyl alkacrylate polymer, alkacrylate polymer, or a combination thereof. In this formulation, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. In this embodiment, preferably, the cellulose ether has an alkyl degree of substitution of 1.3 to 2.0 and hydroxyalkyl molar substitution of up to 0.85. More preferably, the alkyl substitution is methyl. Yet more preferably, the hydroxyalkyl substitution is hydroxpropyl. Most preferably, the cellulose ether is hydroxpropyl methylcellulose. Also, in this embodiment, the alkyl alkacrylate or the alkacrylate polymer has monomeric units of (C₁-C₂₂)alkyl ((C₁-C₁₀)alk)acrylate or (C₁-C₁₀)alkacrylate. Preferably, the alkacrylate polymer is an acrylic polymer or a methacrylic polymer. More preferably, the alkacrylate polymer is ionic acrylic polymer or ionic methacrylic polymer. Yet more preferably, the alkacrylate polymer is a cationic acrylic polymer or cationic methacrylic polymer. Most preferably, the alkacrylate polymer is a copolymer of the acrylic polymer and the methacrylic polymer esters containing quaternary ammonium groups. In this most preferred embodiment, further, the alkacrylate polymer is a copolymer or mixture of copolymers wherein the molar ratio of cationic groups to the neutral esters is in the range of about 1:20 to 1:35 on average.

Yet another embodiment of the present invention provides a non-milled, melt-extruded drug formulation comprising a drug with abuse potential. In this preferred embodiment, the formulation is not snortable via nasal administration. Also, preferably, the drug is an opioid, an amphetamine or methamphetamine. Most preferably, the formulation is directly shaped from the melt-extrudate into a dosage form without (an intermediate) milling step. Also, more preferably, the formulation is directly shaped from the melt-extrudate into a dosage form without (an intermediate) multiparticulating step. Most preferably, the formulation is directly shaped from the melt-extrudate into a dosage form by the process of calendaring.

Another embodiment of the present invention provides a monolithic, non-milled, non-multiparticulated, melt-extruded drug formulation comprising a drug with abuse potential having a diameter from about at least 5.1 mm to about 10 mm and a length from about 5.1 mm to about 30 mm. In this embodiment, preferably, the formulation is directly shaped from the melt-extrudate into a dosage form without (an intermediate) milling step. Further preferably, the formulation is directly shaped from the melt-extrudate into a dosage form without (an intermediate) multiparticulating step. In the above embodiments, most preferably, the formulation is directly shaped from the melt-extrudate into a dosage form by the process of calendaring. Also, as described above, preferably the formulation comprises an abuse-deterrent drug produced by a melt-processed mixture of a) at least one abuse-relevant drug, b) at least one cellulose ether or cellulose ester, and c) at least one alkyl alkacrylate polymer, alkacrylate polymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. Preferably, in this embodiment, the cellulose ether has an alkyl degree of substitution of 1.3 to 2.0 and hydroxyalkyl molar substitution of up to 0.85. Also preferably, the alkyl substitution is methyl. Yet more preferably, the hydroxyalkyl substitution is hydroxpropyl. Most preferably, the cellulose ether is hydroxpropyl methylcellulose. Also in this embodiment, the alkyl alkacrylate or the alkacrylate polymer has monomeric units of (C₁-C₂₂)alkyl ((C₁-C₁₀)alk)acrylate or (C₁-C₁₀)alkacrylate. Preferably, the alkacrylate polymer is an acrylic polymer or a methacrylic polymer. More preferably, the alkacrylate polymer is ionic acrylic polymer or ionic methacrylic polymer. Most preferably, the alkacrylate polymer is a cationic acrylic polymer or cationic methacrylic polymer. In this most preferred embodiment, the alkacrylate polymer is a copolymer of the acrylic polymer and the methacrylic polymer esters containing quaternary ammonium groups. Also, preferably, in this embodiment, the alkacrylate polymer is a copolymer or mixture of copolymers wherein the molar ratio of cationic groups to the neutral esters is in the range of about 1:20 to 1:35 on average.

The present invention provides another embodiment, describing an abuse-deterrent drug formulation formed by a process comprising melt extruding the formulation having at least one therapeutic drug and directly shaping the extrudate into a dosage form without (an intermediate) milling step or multiparticulating step. In this embodiment preferably, the therapeutic drug comprises an abuse-deterrent drug having: a) at least one abuse-relevant drug, b) at least one cellulose ether or cellulose ester, and c) at least one alkyl alkacrylate polymer, alkacrylate polymer, or a combination thereof. In this formulation, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. For this formulation, the cellulose ether has an alkyl degree of substitution of 1.3 to 2.0 and hydroxyalkyl molar substitution of up to 0.85. Preferably, the alkyl substitution is methyl. More preferably, the hydroxyalkyl substitution is hydroxpropyl. And most preferably, the cellulose ether is hydroxpropyl methylcellulose. Also in this embodiment, the alkyl alkacrylate or the alkacrylate polymer has monomeric units of (C₁-C₂₂)alkyl ((C₁-C₁₀)alk)acrylate or (C₁-C₁₀)alkacrylate. More preferably, the alkacrylate polymer is an acrylic polymer or a methacrylic polymer. Also, more preferably, the alkacrylate polymer is ionic acrylic polymer or ionic methacrylic polymer. Yet more preferably, the alkacrylate polymer is a cationic acrylic polymer or cationic methacrylic polymer. And most preferably, the alkacrylate polymer is a copolymer of the acrylic polymer and the methacrylic polymer esters containing quaternary ammonium groups. In this preferred embodiment, the alkacrylate polymer is a copolymer or mixture of copolymers wherein the molar ratio of cationic groups to the neutral esters is in the range of about 1:20 to 1:35 on average.

Another embodiment of the present invention provides a process for the manufacture of an abuse-resistant drug dosage formulation comprising melt extruding a formulation comprising at least one therapeutic drug further comprising directly shaping the extrudate into a dosage form without (an intermediate) milling step or multiparticulating step. In this process preferably, the melt-extrudate comprises a cellulose derivative. More preferably, this cellulose derivative comprises a commercially available Eudragit polymer. Yet more preferably, the melt-extrudate comprises Eudragit® L or Eudragit® RS or both. Most preferably, the melt-extrudate comprises Eudragit® RL or mixtures containing both Eudragit® RS and Eudragit® RL.

In another embodiment, the melt-extrudate comprises an abuse-deterrent drug having: a) at least one abuse-relevant drug, b) at least one cellulose ether or cellulose ester, and c) at least one alkyl alkacrylate polymer, alkacrylate polymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. Preferably, in this embodiment, the cellulose ether has an alkyl degree of substitution of 1.3 to 2.0 and hydroxyalkyl molar substitution of up to 0.85. More preferably, the alkyl substitution is methyl. Yet more preferably, the hydroxyalkyl substitution is hydroxpropyl. Most preferably, the cellulose ether is hydroxpropyl methylcellulose. As also described above, in this embodiment, the alkyl alkacrylate or the alkacrylate polymer has monomeric units of (C₁-C₂₂)alkyl ((C₁-C₁₀)alk)acrylate or (C₁-C₁₀)alkacrylate. Preferably, the alkacrylate polymer is an acrylic polymer or a methacrylic polymer. More preferably, the alkacrylate polymer is ionic acrylic polymer or ionic methacrylic polymer. And most preferably, the alkacrylate polymer is a cationic acrylic polymer or cationic methacrylic polymer. In this most preferred embodiment, the alkacrylate polymer is a copolymer of the acrylic polymer and the methacrylic polymer esters containing quaternary ammonium groups. Also in this most preferred embodiment, the alkacrylate polymer is a copolymer or mixture of copolymers wherein the molar ratio of cationic groups to the neutral esters is in the range of about 1:20 to 1:35 on average.

Yet another embodiment of the present invention provides a monolithic, non-milled, melt-extruded drug formulation comprising a drug with abuse potential wherein the monolithic formulation has a substantially similar drug release profile to a crushed form of the monolithic formulation wherein the monolithic formulation is crushed at about 20,000 rpm to about 50,000 rpm in a coffee grinding machine for about 60 seconds. Preferably, in this embodiment, the melt-extrudate comprises an abuse-deterrent drug having: a) at least one abuse-relevant drug, b) at least one cellulose ether or cellulose ester, and c) at least one alkyl alkacrylate polymer, alkacrylate polymer, or a combination thereof. In this formulation, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. Preferably the cellulose ether has an alkyl degree of substitution of 1.3 to 2.0 and hydroxyalkyl molar substitution of up to 0.85. More preferably, the alkyl substitution is methyl. Also more preferably, the hydroxyalkyl substitution is hydroxpropyl. Most preferably, the cellulose ether is hydroxpropyl methylcellulose. Moreover, in this embodiment, the alkyl alkacrylate or the alkacrylate polymer has monomeric units of (C₁-C₂₂)alkyl ((C₁-C₁₀)alk)acrylate or (C₁-C₁₀)alkacrylate. Preferably, the alkacrylate polymer is an acrylic polymer or a methacrylic polymer. More preferably, the alkacrylate polymer is ionic acrylic polymer or ionic methacrylic polymer. Yet more preferably, the alkacrylate polymer is a cationic acrylic polymer or cationic methacrylic polymer. Most preferably, the alkacrylate polymer is a copolymer of the acrylic polymer and the methacrylic polymer esters containing quaternary ammonium groups. In this most preferred embodiment, the alkacrylate polymer is a copolymer or mixture of copolymers wherein the molar ratio of cationic groups to the neutral esters is in the range of about 1:20 to 1:35 on average. Further in certain preferred embodiments, the drug formulation does not comprise more than 0.5% of a genotoxic compound derived from the abuse relevant drug or another active pharmaceutical ingredient included in the formulation. For example, it has been found that polyethylene oxide oxidizes some opioids to form an N-oxide derivative that might be genotoxic. Accordingly, in embodiments of the invention containing polyethylene oxide or other polymers or substances that cause significant oxidation of opioids, other abuse relevant drugs, or oxidizable non-abuse relevant drugs, then the inventive formulation preferably comprises a sufficient quantity of anti-oxidants to prevent the accumulation of potentially genotoxic derivatives, preferably less than 1%, more preferably less than 0.5%, yet more preferably less than 0.3%, even more preferably less than 0.1%, and most preferably less than 0.05%, by weight of the genotoxic compound as a total of the weight of the drug incorporated into the formulation.

Another embodiment of the present invention provides an abuse-deterrent drug formulation comprising a melt-processed mixture of a) at least one abuse-relevant drug, b) at least one rate altering pharmaceutically acceptable polymer, copolymer, or a combination thereof. In this embodiment, the amount of the drug that is extracted from the formulation by 40% aqueous ethanol within one hour at 37° C. is less than or equal to twice the amount of the drug that is extracted by 0.01 N hydrochloric acid within one hour at 37° C.; and the drug formulation is adapted so as to be useful for oral administration to a human 3, 2, or 1 times daily. Preferably, the rate altering polymer is a cellulose ether or a cellulose ester polymer. In another embodiment, the rate altering polymer is selected from a group consisting of homopolymers, copolymers, or combinations of monomers of N-vinyl lactams, nitrogen-containing monomers, oxygen-containing monomers, vinyl alcohol, ethylene glycol, alkylene oxides, ethylene oxide, propylene oxide, acrylamide, vinyl acetate, hydroxy acid. In yet another embodiment, the rate altering polymer is hydrogen-peroxide polyvinylpyrrolidone polymer. In another preferable embodiment, the rate altering polymer, copolymer, or a combination thereof comprises at least one alkyl alkacrylate polymer, alkacrylate polymer, or a combination thereof. More preferably, the cellulose ether has an alkyl degree of substitution of 1.3 to 2.0 and hydroxyalkyl molar substitution of up to 0.85. Also, more preferably, the alkyl substitution is methyl. Yet more preferably, the hydroxyalkyl substitution is hydroxpropyl. Most preferably, the cellulose ether is hydroxpropyl methylcellulose. In another embodiment, the alkyl alkacrylate or the alkacrylate polymer has monomeric units of (C₁-C₂₂)alkyl ((C₁-C₁₀)alk)acrylate or (C₁-C₁₀)alkacrylate. More preferably, the alkacrylate polymer is an acrylic polymer or a methacrylic polymer. Yet more preferably, the alkacrylate polymer is ionic acrylic polymer or ionic methacrylic polymer. Most preferably, the alkacrylate polymer is a cationic acrylic polymer or cationic methacrylic polymer. Further, in a most preferable embodiment, the alkacrylate polymer is a copolymer of the acrylic polymer and the methacrylic polymer esters containing quaternary ammonium groups. In this most preferable embodiment, the alkacrylate polymer is a copolymer or mixture of copolymers wherein the molar ratio of cationic groups to the neutral esters is in the range of about 1:20 to 1:35 on average. Rate altering polymers may be useful in forming the matrix of the sustained release pharmaceutically acceptable polymers.

Another embodiment of the present invention provides an abuse-deterrent drug formulation comprising a melt-processed mixture of a) at least one abuse-relevant drug, wherein said drug is hydrocodone; b) at least one viscosity altering agent, and c) at least one sustained release polymer, copolymer, or a combination thereof. In this embodiment, more than 30% of the hydrocodone is extracted from the formulation at about one hour at 37° C. in 0.01 N hydrochloric acid; and the drug formulation is adapted so as to be useful for oral administration to a human 3, 2, or 1 times daily. In this embodiment, viscosity altering agents are pharmaceutically acceptable polymers that may be used to alter the viscosity or the glass transition temperature of the polymer melt that is used for the sustained release formulation. In one preferred embodiment, the viscosity altering agent is a cellulose ether or a cellulose ester. In another preferred embodiment, the sustained release polymer, copolymer, or a combination thereof comprises at least one alkyl alkacrylate polymer, alkacrylate polymer, or a combination thereof. Also, preferably, in this embodiment, the cellulose ether has an alkyl degree of substitution of 1.3 to 2.0 and hydroxyalkyl molar substitution of up to 0.85. In a more preferred embodiment, the alkyl substitution is methyl. In another preferred embodiment, the hydroxyalkyl substitution is hydroxpropyl. Most preferably, the cellulose ether is hydroxpropyl methylcellulose. Also in another embodiment of this invention, the alkyl alkacrylate or the alkacrylate polymer has monomeric units of (C₁-C₂₂)alkyl ((C₁-C₁₀)alk)acrylate or (C₁-C₁₀)alkacrylate. Preferably, the alkacrylate polymer is an acrylic polymer or a methacrylic polymer. Yet preferably, the alkacrylate polymer is ionic acrylic polymer or ionic methacrylic polymer. More preferably, the alkacrylate polymer is a cationic acrylic polymer or cationic methacrylic polymer. Most preferably, the alkacrylate polymer is a copolymer of the acrylic polymer and the methacrylic polymer esters containing quaternary ammonium groups. In this most preferred embodiment, the alkacrylate polymer is a copolymer or mixture of copolymers wherein the molar ratio of cationic groups to the neutral esters is in the range of about 1:20 to 1:35 on average.

Another embodiment of the present invention provides an abuse-deterrent drug formulation comprising a melt-processed mixture of a) at least one abuse-relevant drug, wherein said drug is hydrocodone or hydrocodone bitartrate pentahemihydrate, b) at least one cellulose ether or cellulose ester, and c) at least one acrylic polymer, methacrylic polymer, or a combination thereof. In this embodiment, the drug formulation is adapted so as to be useful for oral administration to a human 3, 2, or 1 times daily; and where about ninety percent of the hydrocodone is released in vitro at about 4-6 hours when adapted to be administered 3 times a day, at about 6-10 hours when adapted to be administered 2 times a day and about 16-22 hours when adapted to be administered 1 time a day. In one aspect of this invention, more than 30% of the hydrocodone is extracted from the formulation at about one hour at 37° C. in 0.01 N hydrochloric acid. In another aspect of the formulation, less than 30% of the hydrocodone is extracted from the formulation at about one hour at 37° C. in 0.01 N hydrochloric acid.

Another embodiment of the present invention provides an abuse-deterrent drug formulation comprising a melt-processed mixture of a) at least one abuse-relevant drug, wherein said drug is an opioid; and b) at least one rate altering pharmaceutically acceptable polymer, copolymer, or a combination thereof. In this embodiment, the amount of the drug that is extracted from the formulation by 40% aqueous ethanol within one hour at 37° C. is about 70% to about 110% of the amount of the drug that is extracted by 0.01 N hydrochloric acid within one hour at 37° C.; and the drug formulation is adapted so as to be useful for oral administration to a human 3, 2, or 1 times daily. Also, in another aspect, the amount of the drug that is extracted from the formulation by 40% aqueous ethanol within one hour at 37° C. is about 70% to about 100% of the amount of the drug that is extracted by 0.01 N hydrochloric acid within one hour at 37° C. In yet another aspect, the amount of the drug that is extracted from the formulation by 40% aqueous ethanol within one hour at 37° C. is about 70% to about 90% of the amount of the drug that is extracted by 0.01 N hydrochloric acid within one hour at 37° C. In yet another preferred aspect, the amount of the drug that is extracted from the formulation by 40% aqueous ethanol within one hour at 37° C. is about 75% to about 90% of the amount of the drug that is extracted by 0.01 N hydrochloric acid within one hour at 37° C. Preferably, in this embodiment, the abuse relevant drug further comprise a nonopioid analgesic. The non-opioid anagesic may also be a non-steroidal analgesic, and is optionally further selected from the group consisting of acetaminophen, aspirin, fentaynl, ibuprofen, indomethacin, ketorolac, naproxen, phenacetin, piroxicam, sufentanyl, sunlindac, and interferon alpha. In another embodiment, the non-opioid analgesic is preferably acetaminophen or ibuprofen. Further, in this embodiment, most preferably, the opioid is hydrocodone, or salts or esters thereof.

The inventive formulation preferably is adapted to provide a biphasic rate of release of the abuse when exposed to a suitable aqueous medium in vitro in a USP Type II apparatus. Each phase of the biphasic in vitro rate of release is more preferably zero order or ascending for at least about 4 hours when the formulation is adapted to be suitable for administration to a human every 8 hours (i.e., 3 times per day), for at least about 7 hours when the formulation is adapted to be suitable for administration to a human every 12 hours (i.e., 2 times per day), and for at least 16 hours when the formulation is adapted to be suitable for administration to a human every 24 hours (i.e., 1 time per day).

The inventive formulation preferably releases at least 30-45% of the opioid in about 1 hour in vitro, particularly when the formulation is adapted to be suitable for administration to a human every 12 hours (i.e., 2 times per day). Similarly, the formulation preferably releases at least 90% of the opioid the formulation in about 6 hours to about 9 or about 10 hours both in vitro in a USP Type II Apparatus, or in vivo (with respect to the mean) when administered to a population of healthy North Americans or Western Europeans, particularly when the formulation is adapted to be suitable for, or intended for, administration to a human every 12 hours as needed. However, when the formulation is adapted to be suitable for, or intended for, administration to a human every 24 hours as needed, then the formulation preferably releases at least 90% of the opioid from the formulation in about 15 hours to about 20 hours in vitro (in a USP Type II apparatus) or on average when observed in vivo after administration to an a population of healthy North Americans or Western Europeans, particularly when the formulation is adapted to be suitable for, or intended for, administration to a human every 24 hours as needed.

The inventive formulation preferably provides for relatively complete delivery of the abuse relevant drug. In an embodiment, the inventive formulation releases at least 95% of the opioid in from about 6 hours or 7 hours to about 9 hours or 10 hours after introduction to a USP Type II apparatus. The inventive formulation optionally delivers at least 99% is of the opioid in less than about 12 hours, and optionally in about 10 hours to about 11 hours.

The inventive formulation also preferably provides relatively rapid onset of analgesia, which is preferred for the treatment of moderate to moderately severe pain in humans. Accordingly, the formulation preferably is adapted to provide an AUC for the abuse relevant drug of from about 0.22 to about 0.51 in the first hour after administration, of from about 1.07 to about 1.76 in the second hour after administration, of from about 2.06 to about 3.08 in the third hour after administration, and of from about 3.12 to about 4.44 in the fourth hour after administration, wherein the AUC is determined as the mean value observed in a population of at least 15 healthy North American or Western European people. Values of AUC are measured in ng*h/ml of plasma/mg of hydrocodone. Values of /mg of hydrocodone ignores the weight of salts and hydration and refers only to the weight of the hydrocodone moiety for reference, 15 mg of hydrocodone bitartrate pentahemihydrate is equal to 9.08 mg of free hydrocodone. Also concentration of hydrocodone in 1 h is from about 0.70 to about 1.21 ng/ml of plasma/mg of hydrocodone. Concentration of hydrocodone in 2 h is from about 0.91 to about 1.30 ng/ml of plasma/mg of hydrocodone. Concentration of hydrocodone at 3 h is from about 0.99 to about 1.35 ng/ml of plasma/mg of hydrocodone. Concentration of hydrocodone at 4 h is from about 1.07 to about 1.43 ng/ml of plasma/mg of hydrocodone.

The inventive formulation can contain hydrocodone, and if so, is preferably adapted to produce a mean plasma profile in a normal population of at least 10 healthy North American or Western European residents characterized by a Cmax for hydrocodone of between about 0.4 ng/mL/mg to about 1.9 ng/mL/mg, and more preferably of between about 0.6 ng/mL/mg to about 1.4 ng/mL/mg, and optionally of between about 0.6 ng/mL/mg to about 1.0 ng/mL/mg after a single dose suitable for the treatment of moderate to moderately severe pain for about 12 hours. When the inventive formulation contains hydrocodone the formulation preferably also produces a plasma profile characterized by a Cmin for hydrocodone of between about 0.6 ng/mL/mg to about 1.4 ng/mL/mg after a single dose after a single dose suitable for the treatment of moderate to moderately severe pain for about 12 hours. Moreover, the inventive formulation, in embodiments containing hydrocodone can produce desirable total exposures of the patient's blood plasma to hydrocodone. For example, the inventive formulation can be adapted to produce a minimum AUC for hydrocodone of about 7.0 ng*hr/mL/mg, or optionally about 9.1 ng*hr/mL/mg, to a maximum AUC for hydrocodone of about 19.9 ng*hr/mL/mg, or optionally of about 26.2 ng*hr/mL/mg.

In another embodiment, the present invention also provides a method for treating pain in a human patient, comprising orally administering to the human patient, a formulation described in any of the above embodiments or examples provided below.

The following examples will serve to further illustrate the invention without limiting it. In these examples, “UpM” or “rpm” refers to revolutions per minute, and “h” refers to hours. The term “hydrocodone” in the examples of the different formulation compositions refer to hydrocodone bitartrat pentahemihydrate which was used as the raw material in all of the following formulation composition examples.

### EXAMPLE I

### Dissolution in HCl and Aqueous Ethanol

Following is a description of exemplary methodology for studying rate of dissolution of certain compositions in HCl and 20% aqueous ethanol. Similar methodology may be used for studying rate of dissolution in 40% aqueous ethanol.

(i) Method Description: Dissolution in 0.01 N HCl

Apparatus: USP Dissolution Apparatus II (Paddle)

Rotation speed: 50 rpm

### Media: 0.01 N HCl

Media volume: 900 mL

### Temperature: 37° C.

Sampling time: 1/2/3/4/6/8 hours
Sample volume: 10 mL (no volume replacement)
Sample preparation: used as is
Analytical finish: UV detection, wavelength 280 nm
(ii) Method Description: Dissolution in 20 or 40% Aqueous Ethanol

Apparatus: USP Dissolution Apparatus II (Paddle)

Rotation speed: 50 rpm

### Media: 20 or 40% aqueous ethanol

### Media volume 500 mL

### Temperature: 37° C.

Sampling time: 15/30/45/60/90/120/180/240/360/420/480 minutes
Sample volume: 10 mL (no volume replacement)
Sample preparation: dilution 1+1 with 20% or 40% aqueous ethanol
Analytical finish: UV detection, wavelength 280 nm

### EXAMPLE II

Various compositions of certain formulations are discussed in the following sections.

(i) The composition of certain investigated formulations 1-6 is summarized in Table 1. The formulations do not contain a drug that is subject to abuse; they are presented as proof-of-concept:

TABLE 1
Composition of investigated formulations

| | Formulation No. | | | | | |
|---|---|---|---|---|---|---|
| | Form 1 | Form 2 | Form 3 | Form 4 | Form 5 | Form 6 |
| Preparation | acetaminophen 500 mg Extrudate Tablet | | | | | |
| Composition | 55% acetaminophen | 55% acetaminophen | 55% acetaminophen | 55% acetaminophen | 55% acetaminophen | 55% acetaminophen |
| | 44% Eudragit | 22% Eudragit | 22% Eudragit | 44% Eudragit | 11% Eudragit | 22% Eudragit |
| | RL-PO | RL-PO | RL-PO | RS-PO | RL-PO | RL-PO |
| | 1% colloidal silicon | 22% Eudragit | 22% Methocel | 1% colloidal silicon | 11% Methocel | 22% Klucel EF* |
| | dioxide | RS-PO | K100M | dioxide | K100M | 1% colloidal silicon |
| | | 1% colloidal silicon | 1% colloidal silicon | | 22% Klucel EF* | dioxide |
| | | dioxide | dioxide | | 1% colloidal silicon | |
| | | | | | dioxide | |
| Target weight (mg) | 833 mg | 833 mg | 833 mg | 833 mg | 833 mg | 833 mg |

*Klucel EF: hydroxypropylcellulose

In an embodiment of the invention, a crushed, multiparticulated or powdered mixture of the ingredients may be fed into a co-rotating twin-screw extruder. In one preferred embodiment, a homogeneous powdery mixture of the ingredients was fed into a co-rotating twin-screw extruder (screw diameter 18 mm). Extrusion was carried out at 134° C. (melt temperature in the extruder die transient section) with the screws rotating at 114 rpm and a throughput of 1.5 kg per hour. A slightly off-colored extrudate was obtained and this extrudate was fed into a calendar to form elongated tablets weighing approximately 910 mg. The tablets were cooled to room temperature, i.e. about 25° C.

The dissolution behavior of the tablets was tested in 0.01 N HCl and 20% aqueous ethanol according to the protocol given above.

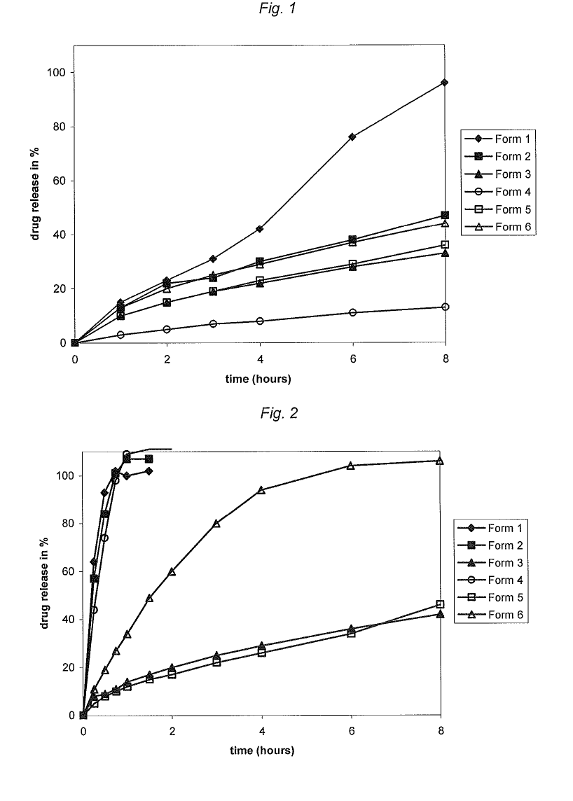

In 0.01 N hydrochloric acid (FIG. 1), Form 1 showed the fastest release of active ingredient with approximately 95% of active ingredient released after 8 hours (note that the 6 hour and 8 hour values showed a high variability). Forms 2 and 6 exhibited a fast initial release of about 20% active ingredient during the first 2 hours followed by a slower, near linear release of another 25% active ingredient over the next 6 hours. The total percentage released active ingredient for Forms 2 and 6 were 47% and 44%, respectively. Forms 3 and 5 showed a near linear release of 33% and 36% active ingredient, respectively, over the complete 8 hours. The slowest release of active ingredient was found in Form 4 (Eudragit RS—PO as only matrix component) with only 13% of the drug released after 8 hours.

The release profiles in 20% aqueous ethanol are shown in FIG. 2. Forms 1, 2 and 4 dissolved rapidly and released the complete amount of active ingredient within the first 45 minutes. Addition of Klucel EF to the matrix as in Form 6 led to a slower but still complete release of active ingredient after approximately 7 hours. The two Methocel K 100M containing extrudates (Form 3 and 5) exhibited by far the slowest release of active ingredient. After 8 hours in 20% aqueous ethanol, Form 3 released 42% of the drug; Form 5 released 46%.

(ii) The composition of the certain other investigated Forms 7-9 is summarized in Table 2:

TABLE 2

| | Formulation No. | | |
|---|---|---|---|
| | Form 7 | Form 8 | Form 9 |
| Composition | 60% acetaminophen | 60% acetaminophen | 60% acetaminophen |
| | 8.0% Eudragit RL-PO | 12.6% Eudragit RL-PO | 8.0% Eudragit RL-PO |
| | 6.0% Methocel K100 | 6.0% Methocel K100 | 6.0% Methocel K100 |
| | 6.0% Methocel K100M | 6.0% Methocel K100M | 6.0% Methocel K100M |
| | 17.2% Kollidon 17PF | 12.6% Xylitol | 17.2% Isomalt F |
| | 1.8% hydrocodone | 1.8% hydrocodone | 1.8% hydrocodone |
| | 1% colloidal silicon | 1% colloidal silicon | 1% colloidal silicon |
| | dioxide | dioxide | dioxide |
| Target weight (mg) | 833.33 | 833.33 | 833.33 |

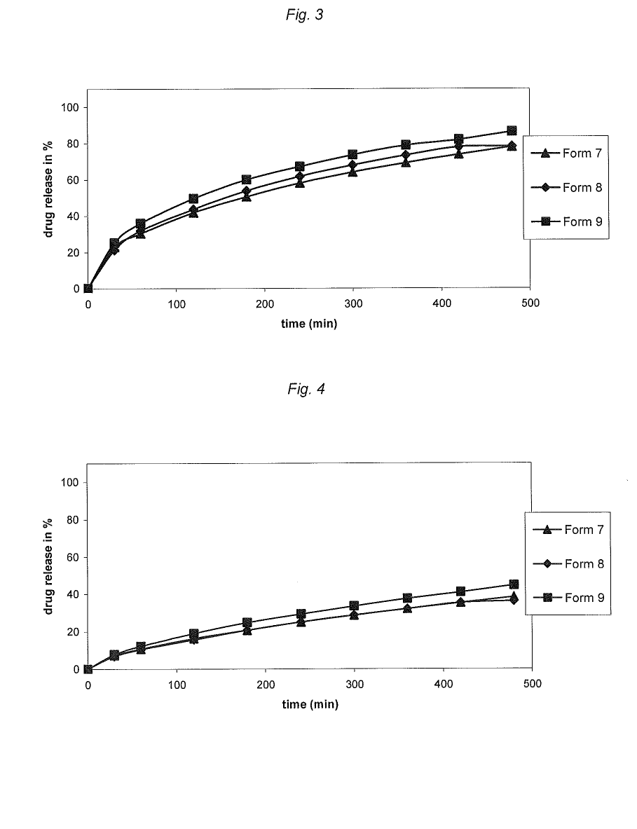

The dissolution behaviour of the tablets was tested in 0.01 N HCl and 40% aqueous ethanol according to the protocol given above. Further, as shown in Table 3 below and in FIG. 3, rate of dissolution of hydrocodone in 0.1 N HCl was measured in various dosage forms 7, 8 and 9 for about 480 minutes.

TABLE 3

| Drug release | Form 7 | Form 8 | Form 9 |
|---|---|---|---|
| testing point (min) | mean in % | mean in % | mean in % |
| 0 | 0 | 0 | 0 |
| 30 | 23 | 21 | 25 |
| 60 | 30 | 32 | 36 |
| 120 | 42 | 44 | 50 |
| 180 | 51 | 54 | 60 |
| 240 | 58 | 62 | 67 |
| 300 | 64 | 68 | 74 |
| 360 | 69 | 73 | 79 |
| 420 | 74 | 78 | 82 |
| 480 | 78 | 78 | 86 |

Also, as shown in Table 4 below and in FIG. 4, rate of dissolution of acetaminophen (APAP) in 0.1 N HCl was measured in various dosage forms 7, 8 and 9 for about 480 minutes.

TABLE 4

| Drug release | Form 7 | Form 8 | Form 9 |
|---|---|---|---|
| testing point (min) | mean in % | mean in % | mean in % |
| 0 | 0 | 0 | 0 |
| 30 | 7 | 7 | 8 |
| 60 | 11 | 11 | 12 |
| 120 | 16 | 16 | 19 |
| 180 | 21 | 21 | 25 |
| 240 | 25 | 25 | 29 |
| 300 | 29 | 29 | 34 |
| 360 | 32 | 32 | 38 |
| 420 | 35 | 35 | 41 |
| 480 | 38 | 36 | 45 |

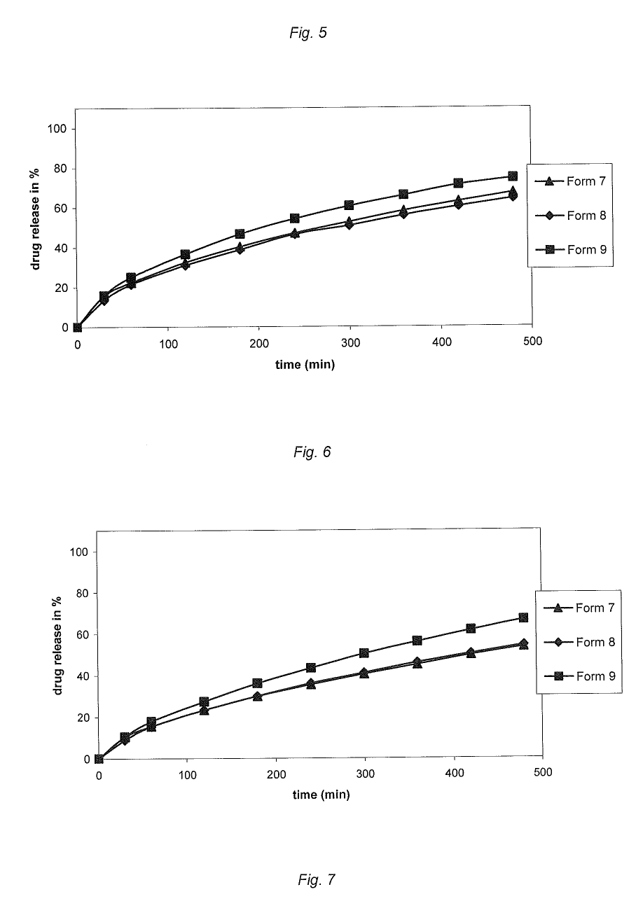

As shown in Table 5 below and in FIG. 5, rate of dissolution of hydrocodone in 40% aqueous ethanol was measured in various dosage forms 7, 8 and 9 for about 480 minutes.

TABLE 5

| Drug release | Form 7 | Form 8 | Form 9 |
|---|---|---|---|
| testing point (min) | mean in % | mean in % | mean in % |
| 0 | 0 | 0 | 0 |
| 30 | 16 | 13 | 16 |
| 60 | 22 | 22 | 25 |
| 120 | 33 | 31 | 37 |
| 180 | 40 | 39 | 47 |
| 240 | 47 | 47 | 54 |
| 300 | 53 | 51 | 61 |
| 360 | 58 | 56 | 66 |
| 420 | 63 | 60 | 71 |
| 480 | 67 | 64 | 75 |

As shown in Table 6 below and in FIG. 6, rate of dissolution of acetaminophen (APAP) in 40% aqueous ethanol was measured in various dosage forms 7, 8 and 9 for about 480 minutes.

TABLE 6

| Drug release | Form 7 | Form 8 | Form 9 |
|---|---|---|---|
| testing point (min) | mean in % | mean in % | mean in % |
| 0 | 0 | 0 | 0 |
| 30 | 10 | 9 | 11 |
| 60 | 16 | 15 | 18 |
| 120 | 23 | 23 | 27 |
| 180 | 30 | 30 | 36 |
| 240 | 36 | 36 | 43 |
| 300 | 41 | 41 | 50 |
| 360 | 45 | 46 | 56 |
| 420 | 50 | 50 | 62 |
| 480 | 54 | 54 | 67 |

Drug release profiles as shown in Tables 3-6 of various dosage form 7, 8 and 9 generally depict that hydrocodone is slowly released in 40% aqueous ethanol (about 10% less drug is released after 8 hours than 0.01 N HCl). Further, drug release of APAP in these formulations is faster in 40% aqueous ethanol than in 0.01 N HCl.

(iii) The composition of Form 31 is summarized in Table 7:

TABLE 7

| | |
|---|---|
| Formulation No. | Form 31 |

APAP/hydrocodone 15/500 mg SR Extrudate Tablet

| | |
|---|---|
| Composition | 60% acetaminophen |
| | 12.6% Eudragit RL-PO |
| | 6.0% Methocel K100 |
| | 6.0% Methocel K100M |
| | 12.6% Xylitol |
| | 1.8% hydrocodone |
| Target weight (mg) | 833.33 |

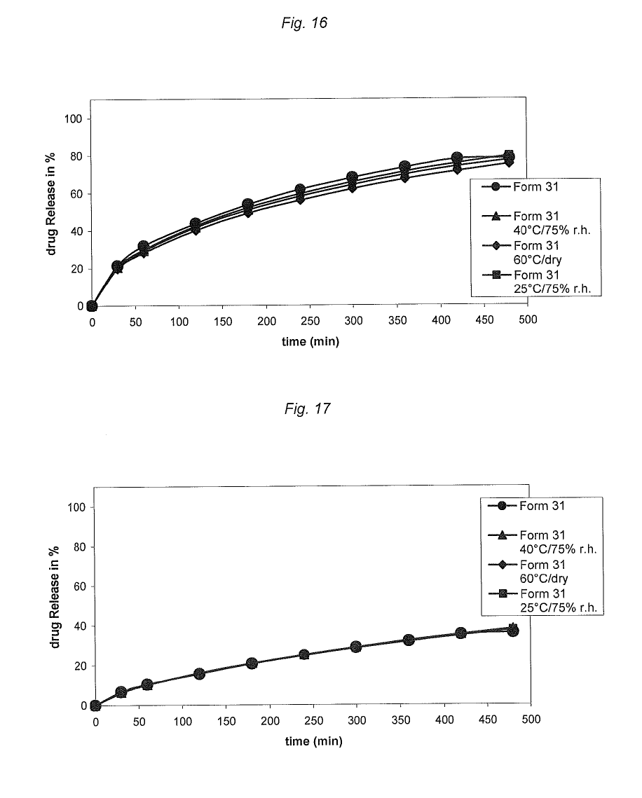

As shown in Table 8 below and in FIG. 16, rate of dissolution of hydrocodone in 0.01 N HCl was measured in dosage form 31 for about 480 minutes directly after manufacturing and after storage for 1 month at 25° C./60% relative humidity, at 40° C./75% relative humidity, and at 60° C. dry, respectively.

As shown in Table 8 below and in FIG. 16, rate of dissolution of hydrocodone in 0.01 N HCl was measured in various dosage forms 31-34 for about 480 minutes.

TABLE 8

| | | Form 31, 1 month | Form 31, 1 month | Form 31, 1 month |
|---|---|---|---|---|
| Drug release | Form 31 | 25° C./60% r.h. | 40° C./75% r.h. | 60° C. dry |
| testing point (min) | mean in % | mean in % | mean in % | mean in % |
| 0 | 0 | 0 | 0 | 0 |
| 30 | 21 | 21 | 20 | 20 |
| 60 | 32 | 30 | 29 | 28 |
| 120 | 44 | 43 | 42 | 40 |
| 180 | 54 | 52 | 51 | 49 |
| 240 | 62 | 60 | 58 | 56 |
| 300 | 68 | 66 | 64 | 62 |
| 360 | 73 | 71 | 70 | 67 |
| 420 | 78 | 76 | 74 | 72 |
| 480 | 78 | 80 | 78 | 75 |

As shown in Table 9 below and in FIG. 17, rate of dissolution of acetaminophen in 0.01 N HCl was measured in dosage form 31 for about 480 minutes directly after manufacturing and after storage for 1 month at 25° C./60% relative humidity, at 40° C./75% relative humidity, and at 60° C. dry, respectively.

TABLE 9

| | | Form 31, 1 month | Form 31, 1 month | Form 31, 1 month |
|---|---|---|---|---|
| Drug release | Form 31 | 25° C./60% r.h. | 40° C./75% r.h. | 60° C. dry |
| testing point (min) | mean in % | mean in % | mean in % | mean in % |
| 0 | 0 | 0 | 0 | 0 |
| 30 | 7 | 6 | 6 | 6 |
| 60 | 11 | 10 | 10 | 10 |
| 120 | 16 | 16 | 16 | 16 |
| 180 | 21 | 21 | 21 | 21 |
| 240 | 25 | 25 | 25 | 25 |
| 300 | 29 | 29 | 29 | 29 |
| 360 | 32 | 32 | 32 | 32 |
| 420 | 35 | 35 | 35 | 35 |
| 480 | 36 | 38 | 38 | 38 |

(iv) The composition of the certain other investigated Forms 32-37 is summarized in

TABLE 10

| | Formulation No. | | | | | |
|---|---|---|---|---|---|---|
| | Form 32 | Form 33 | Form 34 | Form 35 | Form 36 | Form 37 |
| Preparation | acetaminophen 500 mg Extrudate Tablet | | | | | |
| Composition | 60% acetaminophen | 60% acetaminophen | 60% acetaminophen | 60% acetaminophen | 60% acetaminophen | 60% acetaminophen |
| | 13% Eudragit | 13% Eudragit | 6.5% Eudragit | 6.5% Eudragit | 13% Eudragit | 13% Eudragit |
| | RL-PO | RL-PO | RL-PO | RL-PO | RL-PO | RL-PO |
| | 13% Methocel | 13% Methocel | 6.5% Eudragit | 6.5% Eudragit | 13% Methocel | 13% Kollidon VA64 |
| | K100M | K100M | RS-PO | RS-PO | K100M | 13% Klucel EF |
| | 13% Klucel EF | 13% Kollidon VA64 | 26% Klucel EF | 13% Methocel | 13% Polyox | 1% colloidal silicon |
| | 1% colloidal silicon | 1% colloidal silicon | 1% colloidal silicon | K100M | 1% colloidal silicon | dioxide |
| | dioxide | dioxide | dioxide | 13% Kollidon VA64 | dioxide | |
| | | | | 1% colloidal silicon | | |
| | | | | dioxide | | |
| Target weight (mg) | 833 mg | 833 mg | 833 mg | 833 mg | 833 mg | 833 mg |

The dissolution behaviour of the tablets was tested in 0.01 N HCl and 20% aqueous ethanol according to the protocol given above.

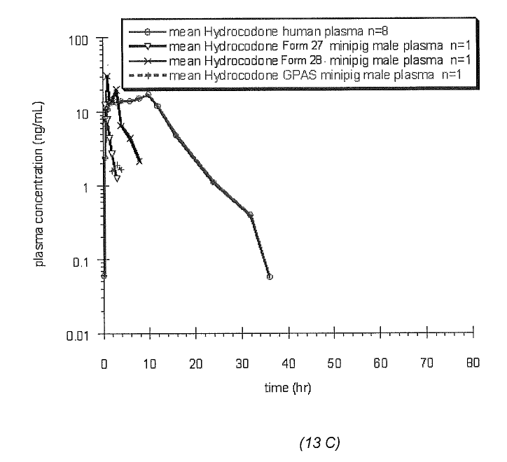

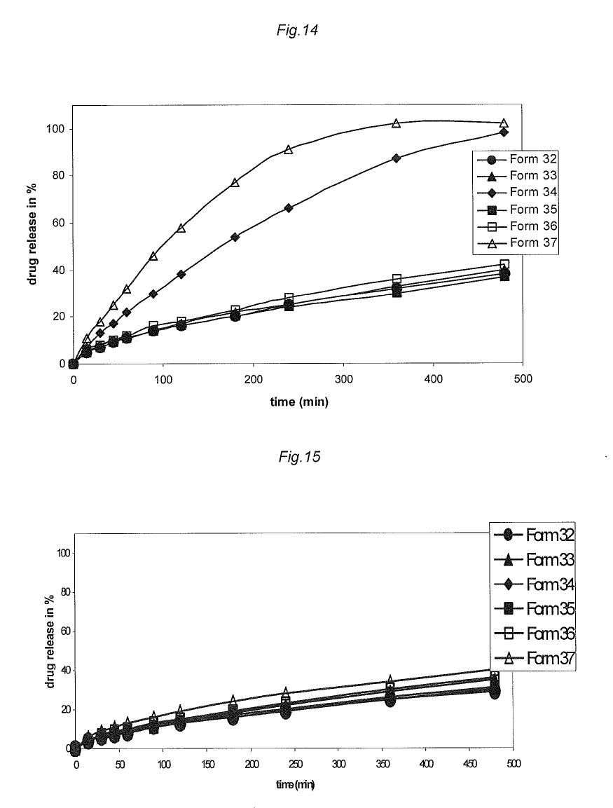

### As shown in Table 11 below and in FIG. 14, rate of dissolution of hydrocodone in 20% aqueous ethanol was measured in various dosage forms 32-37 for about 480 minutes.

TABLE 11

| Drug release | | | | | | |
|---|---|---|---|---|---|---|
| testing | Form 32 | Form 33 | Form 34 | Form 35 | Form 36 | Form 37 |
| point (min) | mean in % | mean in % | mean in % | mean in % | mean in % | mean in % |
| 0 | 0 | 0 | 0 | 0 | 0 | 0 |
| 15 | 5 | 5 | 7 | 5 | 6 | 11 |
| 30 | 7 | 8 | 13 | 7 | 8 | 18 |
| 45 | 9 | 10 | 17 | 9 | 10 | 25 |
| 60 | 11 | 11 | 22 | 11 | 12 | 32 |
| 90 | 14 | 14 | 30 | 14 | 16 | 46 |
| 120 | 16 | 17 | 38 | 16 | 18 | 58 |
| 180 | 20 | 22 | 54 | 20 | 23 | 77 |
| 240 | 25 | 25 | 66 | 24 | 28 | 91 |
| 360 | 32 | 33 | 87 | 30 | 36 | 102 |
| 480 | 38 | 40 | 98 | 37 | 42 | 102 |

As shown in Table 12 below and in FIG. 15, rate of dissolution of hydrocodone in 0.01 N HCl was measured in various dosage forms 32-37 for about 480 minutes.

TABLE 12

| Drug release | | | | | | |
|---|---|---|---|---|---|---|
| testing point | Form 32 | Form 33 | Form 34 | Form 35 | Form 36 | Form 37 |
| (min) | mean in % | mean in % | mean in % | mean in % | mean in % | mean in % |
| 0 | 0 | 0 | 0 | 0 | 0 | 0 |
| 15 | 4 | 4 | 5 | 4 | 4 | 6 |
| 30 | 6 | 6 | 5 | 6 | 7 | 9 |
| 45 | 7 | 8 | 7 | 7 | 9 | 11 |
| 60 | 8 | 9 | 9 | 8 | 10 | 13 |
| 90 | 11 | 12 | 11 | 11 | 13 | 16 |
| 120 | 13 | 14 | 13 | 13 | 15 | 19 |
| 180 | 16 | 18 | 17 | 17 | 19 | 24 |
| 240 | 19 | 22 | 20 | 20 | 23 | 28 |
| 360 | 25 | 29 | 25 | 26 | 30 | 34 |
| 480 | 29 | 35 | 30 | 31 | 36 | 40 |

Based on the above experiments, it was visually observed that in 20% aqueous ethanol, (i) Form 32 tablets dissolved very slowly, (ii) Form 33 tablets formed a gel-like coating in-part, whereas the remaining portion was unchanged, (iii) Form 34 tablets formed a small tablet core on the paddle bottom, (iv) Form 35 tablets had a substantially intact tablet core with a surrounding transparent fluff, (v) Form 36 tablets had about an 80% intact tablets after 8 h and (vi) For Form 37, Tablets 3, 4, 6 dissolved after 5 h, Tablet 5 dissolved after 6 h, Tablet 2 after 7 h and a small amount of Tablet 1 was left after 8 h. Further, based on the above experiments, it was visually observed that in 0.01 N HCl, (i) Form 32 had about 90% intact tablets after 8 h, with flocculation, (ii) Form 33 had 90% intact tablets after 8 h, with flocculation, (iii) Form 34 had about 90% intact tablets after 8 h, with flocculation, (iv) Form 35 had about 90% intact tablets after 8 h, with flocculation, (v) Form 36 had about 80% intact tablets after 8 h and the outer layer of the tablets were very hackly with flocculation and (vi) Form 37 was substantially unchanged after 8 h. Test Characteristic Results based on the above experiments provided Flexural strength as well as breaking strength, as depicted in Table 13 and 14 below:

TABLE 13

| Flexural | | | | | | |
|---|---|---|---|---|---|---|
| Strength | Form 32 | Form 33 | Form 34 | Form 35 | Form 36 | Form 37 |
| Mean Value (N) | >500 | >500 | >500 | >500 | 431 | >500 |

TABLE 14

| Breaking | | | | | | |
|---|---|---|---|---|---|---|
| Strength | Form 32 | Form 33 | Form 34 | Form 35 | Form 36 | Form 37 |
| Mean Value (N) | >500 | 431 | >500 | 418 | >500 | 484 |

(v) The dissolution behaviour of the tablets of Forms 32, 34 and 36 was tested in 0.01 N HCl+5% NaCl, 0.05 M phosphate buffer pH 6.78/50 rpm, 0.01 N HCl+0.9% NaCl/50 rpm and 0.01 N HCl/200 rpm according to substantially similar protocols as provided above.

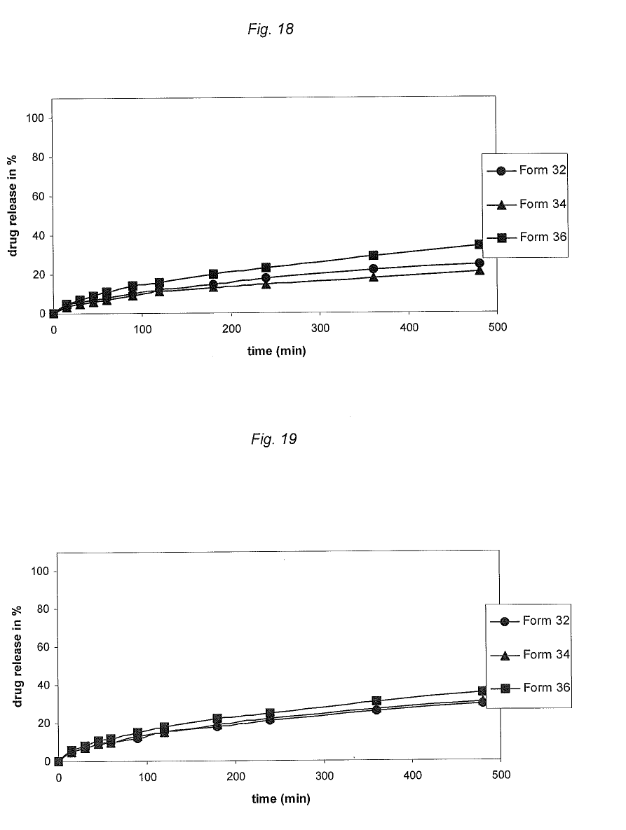

Further, as shown in Table 15 below and in FIG. 18, rate of dissolution of acetaminophen in 0.01 N HCl+5% NaCl was measured in various dosage Forms 32, 34 and 36 for about 480 minutes.

TABLE 15

| Drug release | Form 32 | Form 34 | Form 36 |
|---|---|---|---|
| testing point (min) | mean in % | mean in % | mean in % |
| 0 | 0 | 0 | 0 |
| 15 | 4 | 3 | 5 |
| 30 | 6 | 5 | 7 |
| 45 | 7 | 6 | 9 |
| 60 | 8 | 7 | 11 |
| 90 | 10 | 9 | 14 |
| 120 | 12 | 11 | 16 |
| 180 | 15 | 13 | 20 |
| 240 | 18 | 15 | 23 |
| 360 | 22 | 18 | 29 |
| 480 | 25 | 21 | 34 |

Further, as shown in Table 16 below and in FIG. 19, rate of dissolution of acetaminophen in 0.05 M phosphate buffer pH 6.78/50 rpm was measured in various dosage Forms 32, 34 and 36 for about 480 minutes.

TABLE 16

| Drug release | Form 32 | Form 34 | Form 36 |
|---|---|---|---|
| testing point (min) | mean in % | mean in % | mean in % |
| 0 | 0 | 0 | 0 |
| 15 | 5 | 5 | 6 |
| 30 | 7 | 7 | 8 |
| 45 | 9 | 9 | 11 |
| 60 | 10 | 10 | 12 |
| 90 | 12 | 13 | 15 |
| 120 | 15 | 15 | 18 |
| 180 | 18 | 19 | 22 |
| 240 | 21 | 22 | 25 |
| 360 | 26 | 27 | 31 |
| 480 | 30 | 31 | 36 |

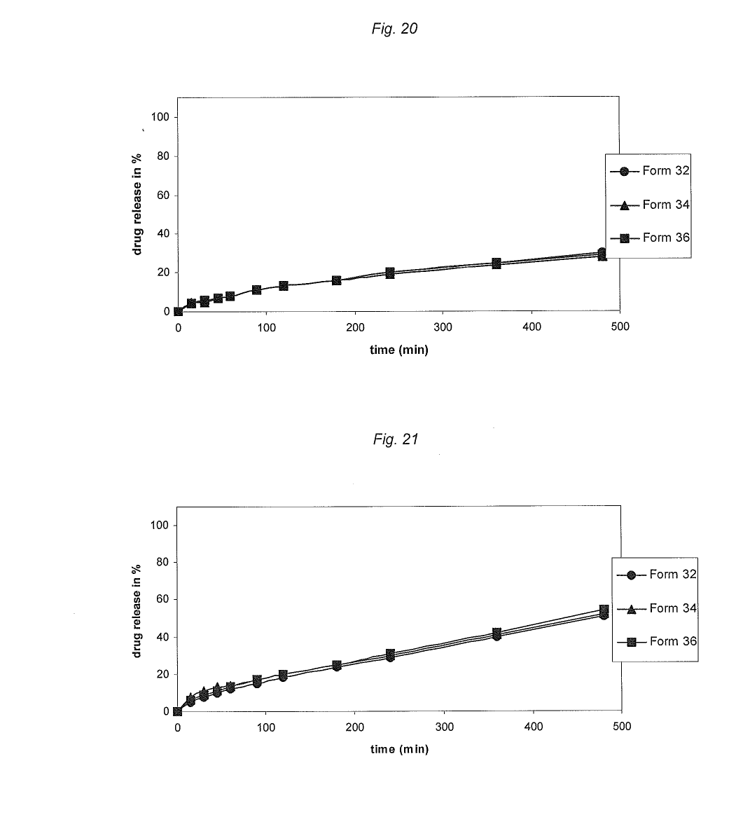

As shown in Table 17 below and in FIG. 20, rate of dissolution of acetaminophen in 0.01 N HCl+0.9% NaCl/50 rpm was measured in various dosage Forms 32, 34 and 36 for about 480 minutes.

TABLE 17

| Drug release | Form 32 | Form 34 | Form 36 |
|---|---|---|---|
| testing point (min) | mean in % | mean in % | mean in % |
| 0 | 0 | 0 | 0 |
| 15 | 4 | 5 | 4 |
| 30 | 6 | 5 | 6 |
| 45 | 7 | 7 | 7 |
| 60 | 8 | 8 | 8 |
| 90 | 11 | 11 | 11 |
| 120 | 13 | 13 | 13 |
| 180 | 16 | 16 | 16 |
| 240 | 20 | 19 | 20 |
| 360 | 25 | 24 | 25 |
| 480 | 30 | 28 | 29 |

As shown in Table 18 below and in FIG. 21, rate of dissolution of acetaminophen in 0.01 N HCl/200 rpm was measured in various dosage Forms 32, 34 and 36 for about 480 minutes.

TABLE 18

| Drug release | Form 32 | Form 34 | Form 36 |
|---|---|---|---|
| testing point (min) | mean in % | mean in % | mean in % |
| 0 | 0 | 0 | 0 |
| 15 | 5 | 8 | 8 |
| 30 | 8 | 11 | 9 |
| 45 | 10 | 13 | 11 |
| 60 | 12 | 14 | 13 |
| 90 | 15 | 17 | 17 |
| 120 | 18 | 20 | 20 |
| 180 | 24 | 25 | 25 |
| 240 | 29 | 30 | 31 |
| 360 | 40 | 41 | 42 |
| 480 | 51 | 52 | 54 |

(vi) The composition of the certain other investigated Forms 38-40 is summarized in Table 19:

TABLE 19

| Formulation No. | Form 38 | Form 39 | Form 40 |
|---|---|---|---|
| Preparation | acetaminophen 500 mg Extrudate Tablet | | |
| Composition | 60% acetaminophen | 60% acetaminophen | 60% acetaminophen |
| | 8.0% Eudragit RL-PO | 12.6% Eudragit RL-PO | 8.0% Eudragit RL-PO |
| | 6.0% Methocel K100 | 6.0% Methocel K100 | 6.0% Methocel K100 |
| | 6.0% Methocel K100M | 6.0% Methocel K100M | 6.0% Methocel K100M |
| | 17.2% Kollidon 17PF | 12.6% Xylitol | 17.2% Isomalt F |
| | 1.8% hydrocodone | 1.8% hydrocodone | 1.8% hydrocodone |
| | 1% colloidal silicon dioxide | 1% colloidal silicon dioxide | 1% colloidal silicon dioxide |
| Target weight (mg) | 833.33 | 833.33 | 833.33 |

The dissolution behaviour of the tablets of Forms 38, 39 and 40 was tested in 0.01 N HCl and 40% aqueous ethanol according to protocols as provided above.

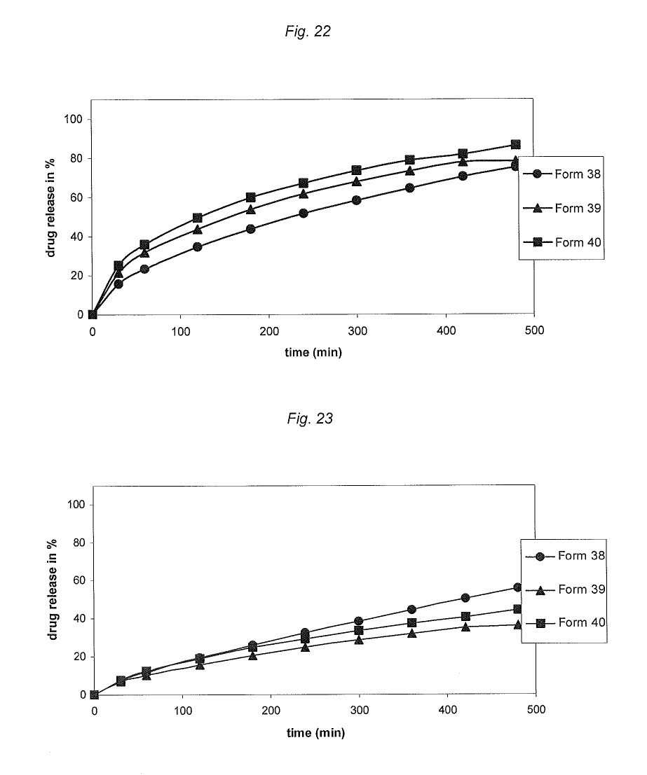

As shown in Table 20 below and in FIG. 22, rate of dissolution of hydrocodone in 0.01 N HCl was measured in various dosage Forms 38, 39 and 40 for about 480 minutes.

TABLE 20

| Drug release | Form 38 | Form 39 | Form 40 |
|---|---|---|---|
| testing point (min) | mean in % | mean in % | mean in % |
| 0 | 0 | 0 | 0 |
| 30 | 16 | 21 | 25 |
| 60 | 23 | 32 | 36 |
| 120 | 35 | 44 | 50 |
| 180 | 44 | 54 | 60 |
| 240 | 52 | 62 | 67 |
| 300 | 58 | 68 | 74 |
| 360 | 65 | 73 | 79 |
| 420 | 71 | 78 | 82 |
| 480 | 75 | 78 | 86 |

As shown in Table 21 below and in FIG. 23, rate of dissolution of acetaminophen (APAP) in 0.01 N HCl was measured in various dosage Forms 38, 39 and 40 for about 480 minutes.

TABLE 21

| Drug release | Form 38 | Form 39 | Form 40 |
|---|---|---|---|
| testing point (min) | mean in % | mean in % | mean in % |
| 0 | 0 | 0 | 0 |
| 30 | 8 | 7 | 8 |
| 60 | 12 | 11 | 12 |
| 120 | 20 | 16 | 19 |
| 180 | 26 | 21 | 25 |
| 240 | 33 | 26 | 29 |
| 300 | 39 | 29 | 34 |
| 360 | 44 | 32 | 38 |
| 420 | 50 | 35 | 41 |
| 480 | 56 | 36 | 46 |

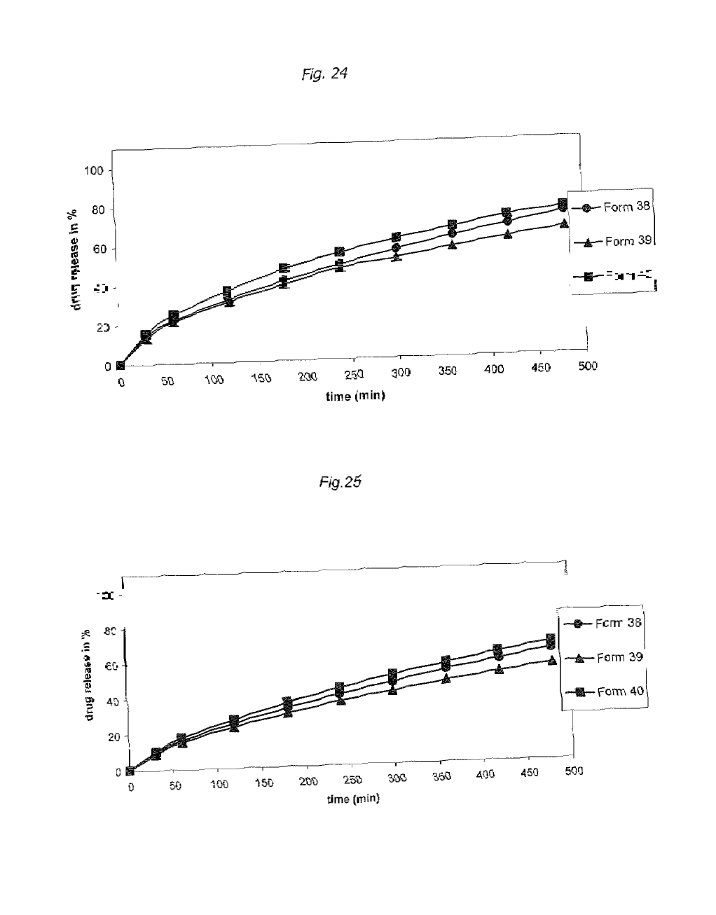

As shown in Table 22 below and in FIG. 24, rate of dissolution of hydrocodone in 40% aqueous ethanol was measured in various dosage Forms 38, 39 and 40 for about 480 minutes.

TABLE 22

| Drug release | Form 38 | Form 39 | Form 40 |
|---|---|---|---|
| testing point (min) | mean in % | mean in % | mean in % |
| 0 | 0 | 0 | 0 |
| 30 | 15 | 13 | 16 |
| 60 | 22 | 22 | 25 |
| 120 | 32 | 31 | 37 |
| 180 | 41 | 39 | 47 |
| 240 | 48 | 47 | 54 |
| 300 | 55 | 51 | 61 |
| 360 | 62 | 56 | 66 |
| 420 | 67 | 60 | 71 |
| 480 | 72 | 64 | 75 |

As shown in Table 23 below and in FIG. 25, rate of dissolution of acetaminophen (APAP) in 40% aqueous ethanol was measured in various dosage Forms 38, 39 and 40 for about 480 minutes.

TABLE 23

| Drug release | Form 38 | Form 39 | Form 40 |
|---|---|---|---|
| testing point (min) | mean in % | mean in % | mean in % |
| 0 | 0 | 0 | 0 |
| 30 | 10 | 9 | 11 |
| 60 | 16 | 15 | 18 |
| 120 | 25 | 23 | 27 |
| 180 | 33 | 30 | 36 |
| 240 | 40 | 36 | 43 |
| 300 | 46 | 41 | 50 |
| 360 | 52 | 46 | 56 |
| 420 | 58 | 50 | 62 |
| 480 | 63 | 54 | 67 |

### EXAMPLE III

### Method for Determining Breaking Strength of Tablets:

An oblong tablet having a diameter from about 5.1 mm to about 10 mm and length from about 5.1 mm to about 30 mm is placed flat in the tablet holder so that the seam is facing up (away from the wedge), i.e. the breaking strength is measured against the seam. The wedge-shaped cylinder is pushed perpendicular to the long side of the tablet as depicted in FIG. 7 and moves into the tablet at a constant speed until the tablet breaks. The force needed to break the tablet is recorded. The maximum force applicable is 500 Newton.

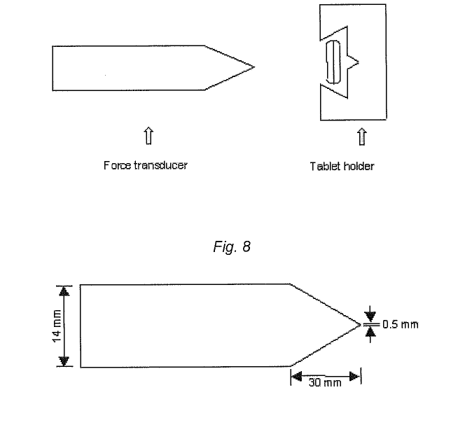

The apparatus used for the measurement is a “Pharma Test PTB 501” hardness tester, Fmax=500 N, draw max. 40 mm, forward speed 3 mm/s. Measurements were performed using a cylinder (diameter 14 mm) with a wedge-shaped tip with dimensions depicted in FIG. 8. (All apparatus from Pharma Test Apparatebau, Hainburg, Germany).

Following compositions of certain investigated Forms 10-18 are illustrative of various dosage form having varying strength:

### I. Tablets with Breaking Strengths Greater than 150 N:

| Form 10 | Form 11 |
|---|---|
| 60% acetaminophen | 60% acetaminophen |
| 8.0% Eudragit RL-PO | 8.0% Eudragit RL-PO |
| 6.0% Methocel K100 | 6.0% Methocel K100 |
| 6.0% Methocel K100M | 6.0% Methocel K100M |
| 17.2% Xylit | 17.2% Isomalt F |
| 1.8% hydrocodone | 1.8% hydrocodone |
| 1% colloidal silicon dioxide | 1% colloidal silicon dioxide |

The breaking strength for Forms 10 is about 190 N, whereas the breaking strength for Form 11 is about 250 N.

### II. Tablets with Breaking Strengths Greater than 300 N:

| Form 12 | Form 13 |
|---|---|
| 60% acetaminophen | 60% acetaminophen |
| 10.1% Eudragit RL-PO | 11.4% Klucel EF |
| 6% Methocel K100 | 11.4% Eudragit RL-PO |
| 6% Methocel K100M | 11.4% Methocel K100 |
| 10.1% Klucel EF | 3% Lutrol F68 |
| 5% Plurol Oleique CC | 1.8% hydrocodone |
| 1.8% hydrocodone | 1% colloidal silicon dioxide |
| 1% colloidal silicon dioxide | |

The breaking strength for Form 12 is about 339 N, whereas the breaking strength for Form 13 is about 410 N.

III. Tablets with breaking strengths greater than 450 N:

| Form 14 | Form 15 |
|---|---|
| 60% acetaminophen | 60% acetaminophen |
| 19.2% Kollidon VA64 | 12.6% Eudragit RL-PO |
| 9% Eudragit RL-PO | 6.0% Methocel K100 |
| 9% Methocel K100 | 6.0% Methocel K100M |
| 1.8% hydrocodone | 12.6% Xylit |
| 1% colloidal silicon dioxide | 1.8% hydrocodone |
| | 1% colloidal silicon dioxide |

The breaking strength for Form 14 is about 454 N, whereas the breaking strength for Form 15 is about 484 N.

IV. Tablets with Breaking Strengths Greater than 500 N:

| Form 16 | Form 17 | Form 18 |
|---|---|---|
| 60% acetaminophen | 60% acetaminophen | 60% acetaminophen |
| 12.6% Eudragit RL-PO | 18.6% Eudragit RL-PO | 18.6% Eudragit RL-PO |
| 6.0% Methocel K100 | 18.6% Methocel K100 | 18.6% Methocel K100 |
| 6.0% Methocel K100M | 1.8% hydrocodone | 1.8% hydrocodone |
| 12.6% Klucel EF | 1% colloidal silicon dioxide | 1% colloidal silicon dioxide |
| 1.8% hydrocodone | | |
| 1% colloidal silicon dioxide | | |

The breaking strength for Forms 16, 17 and 18 is greater than about 500 N.

### EXAMPLE IV

Following compositions of certain investigated Forms 19-22 are illustrative of various dosage form having certain release profiles for hydrocodone, where less than 30% hydrocodone after 1 h in 0.01 N HCl at 37° C.

### Tablets that Release Less than 30% Hydrocodone After 1 h in 0.01 N HCl at 37° C.

In exemplary embodiments the release profile is provided for various dosage forms for intact and crushed tablets in 40% aqueous ethanol and 0.01 N HCl. As shown below in the following examples, in one preferred embodiment for intact tablets, the drug release in the first hour in 40% aqueous ethanol is less than or equal to twice the amount released in 0.01 N HCl. In a more preferred embodiment for intact tablets, the drug release in the first hour in 40% aqueous ethanol is less than or equal to 1.5 times the amount released in 0.01 N HCl. In the most preferred embodiment for intact tablets, the drug release in the first hour in 40% aqueous ethanol is less than or equal to 0.90 the amount released in 0.01 N HCl.

In another preferred embodiment for crushed tablets, the drug release in the first hour in 40% aqueous ethanol is less than or equal to three times the amount released in 0.01 N HCl. In this embodiment, complete release occurs after about 3 or more hours in aqueous 40% alcohol. In a more preferred embodiment for crushed tablets, the drug release in the first hour in 40% aqueous ethanol is less than or equal to 2.5 times the amount released in 0.01 N HCl. In this embodiment, complete release occurs after about 8 or more hours in aqueous 40% alcohol. In the most preferred embodiment for crushed tablets, the drug release in the first hour in 40% aqueous ethanol is less than or equal to twice the amount released in 0.01 N HCl. In this embodiment, complete release occurs after about 8 or more hours in aqueous 40% alcohol.

### Intact Tablets

a.) release after 1 h in 40% ethanol at 37° C. less or equal twice the release in 0.01 N HCl for Form 19, as shown in Table 24:

TABLE 24

| | Drug release | | |
|---|---|---|---|
| | hydrocodone | in 0.01 | in 40% |
| | testing time | N HCl | EtOH |
| Form 19 | point (min) | mean in % | mean in % |
| 60% acetaminophen | 0 | 0 | 0 |
| 19.2% Kollidon VA64 | 30 | 16 | 24 |
| 9% Eudragit RL-PO | 60 | 22 | 44 |
| 9% Methocel K100 | 120 | 32 | 64 |
| 1.8% hydrocodone | 180 | 40 | 79 |
| 1% colloidal silicon dioxide | 240 | 46 | 89 |
| | 300 | 52 | 97 |
| | 360 | 57 | 101 |
| | 420 | 62 | 103 |
| | 480 | 66 | 103 |

b.) release after 1 h in 40% ethanol at 37° C. less or equal 1.5 times the release in 0.01 N HCl for Form 20, as shown in Table 25:

TABLE 25

| | Drug release | | |
|---|---|---|---|
| | hydrocodone | in 0.01 | in 40% |
| | testing time | N HCl | EtOH |
| Form 20 | point (min) | mean in % | mean in % |
| 60% acetaminophen | 0 | 0 | 0 |
| 12.6% Eudragit RL-PO | 30 | 15 | 16 |
| 12.3% Methocel K100 | 60 | 21 | 20 |
| 6% Methocel K100M | 120 | 30 | 28 |
| 6.3% Klucel EF | 180 | 37 | 36 |
| 1.8% hydrocodone | 240 | 43 | 41 |
| 1% colloidal silicon dioxide | 300 | 48 | 48 |
| | 360 | 52 | 53 |
| | 420 | 57 | 58 |
| | 480 | 60 | 62 |

### 2. Crushed Tablets

a.) release after 1 h in 40% ethanol at 37° C. less or equal three times the release in 0.01 N HCl for Form 21, also as shown in Table 26:

TABLE 26

| | Drug release | | |
|---|---|---|---|
| | hydrocodone | in 0.01 | in 40% |
| | testing time | N HCl | EtOH |
| Form 21 | point (min) | mean in % | mean in % |
| 60% acetaminophen | 0 | 0 | 0 |
| 11.4% Klucel EF | 30 | 15 | 53 |
| 11.4% Eudragit RL-PO | 60 | 22 | 64 |
| 11.4% Methocel K100 | 120 | 32 | 83 |
| 3% Lutrol F68 | 180 | 42 | 91 |
| 1.8% hydrocodone | 240 | 50 | 98 |
| 1% colloidal silicon dioxide | 300 | 58 | 100 |
| | 360 | 65 | 101 |
| | 420 | 71 | 101 |
| | 480 | 76 | 101 |

b.) release after 1 h in 40% ethanol at 37° C. less or equal 2.5 times the release in 0.01 N HCl for Form 22, as shown in Table 27:

TABLE 27

| | Drug release | | |
|---|---|---|---|
| | hydrocodone | in 0.01 | in 40% |
| | testing time | N HCl | EtOH |
| Form 22 | point (min) | mean in % | mean in % |
| 60% acetaminophen | 0 | 0 | 0 |
| 10.1% Eudragit RL-PO | 30 | 16 | 45 |
| 6% Methocel K100 | 60 | 23 | 52 |
| 6% Methocel K100M | 120 | 32 | 61 |
| 10.1% Klucel EF | 180 | 40 | 68 |
| 5% Plurol Oleique CC | 240 | 47 | 75 |
| 1.8% hydrocodone | 300 | 53 | 80 |
| 1% colloidal silicon dioxide | 360 | 59 | 84 |
| | 420 | 65 | 88 |
| | 480 | 69 | 91 |

### EXAMPLE V

Following compositions of certain investigated Forms 23-25 are illustrative of various dosage form having certain release profiles for hydrocodone, where more than 30% hydrocodone is released after 1 h in 0.01 N HCl at 37° C.

### Tablets that Release More than 30% Hydrocodone After 1 h in 0.01 N HCl at 37° C.:

In exemplary embodiments the release profile is provided for various dosage forms for intact and crushed tablets in 40% aqueous ethanol and 0.01 N HCl. As shown below in the following examples, in one preferred embodiment for intact tablets, the drug release in the first hour in 40% aqueous ethanol is less than or equal to 1.5 times the amount released in 0.01 N HCl. In the more preferred embodiment for intact tablets, the drug release in the first hour in 40% aqueous ethanol is less than or equal to 0.90 the amount released in 0.01 N HCl.

In another preferred embodiment for crushed tablets, the drug release in the first hour in 40% aqueous ethanol is less than or equal to twice the amount released in 0.01 N HCl.

### 1. Intact Tablets

a.) release after 1 h in 40% ethanol at 37° C. less or equal 1.5 times the release in 0.01 N HCl for Form 23, as shown in Table 28:

TABLE 28

| | Drug release | | |
|---|---|---|---|
| | hydrocodone | in 0.01 | in 40% |
| | testing time | N HCl | EtOH |
| Form 23 | point (min) | mean in % | mean in % |
| 76% aceteminophen | 0 | 0 | 0 |
| 11.2% Eudragit RL-PO | 30 | 24 | 24 |
| 10.0% Methocel K100 | 60 | 34 | 39 |
| 1.8% hydrocodone | 120 | 48 | 61 |
| 1% colloidal silicon dioxide | 180 | 58 | 78 |
| | 240 | 66 | 90 |
| | 300 | 72 | 99 |
| | 360 | 77 | 103 |
| | 420 | 82 | 105 |
| | 480 | 86 | 105 |

b.) release after 1 h in 40% ethanol at 37° C. less or equal 0.9 times the release in 0.01 N HCl, for Form 24, as shown in Table 29:

TABLE 29

| | Drug release | | |
|---|---|---|---|
| | hydrocodone | in 0.01 | in 40% |
| | testing time | N HCl | EtOH |
| Form 24 | point (min) | mean in % | mean in % |
| 60% acetaminophen | 0 | 0 | 0 |
| 8.0% Eudragit RL-PO | 30 | 25 | 16 |
| 6.0% Methocel K100 | 60 | 36 | 25 |
| 6.0% Methocel K100M | 120 | 50 | 37 |
| 17.2% Isomalt F | 180 | 60 | 47 |
| 1.8% hydrocodone | 240 | 67 | 54 |
| 1% colloidal silicon dioxide | 300 | 74 | 61 |
| | 360 | 79 | 66 |
| | 420 | 82 | 71 |
| | 480 | 86 | 75 |

### 2. Crushed Tablets

a.) release after 1 h in 40% ethanol at 37° C. less or equal twice the release in 0.01 N HCl

for Form 25, as shown in Table 30:

TABLE 30

| | Drug release | | |
|---|---|---|---|
| | hydrocodone | in 0.01 | in 40% |
| | testing time | N HCl | EtOH |
| Form 25 | point (min) | mean in % | mean in % |
| 60% acetaminophen | 0 | 0 | 0 |
| 12.6% Eudragit RL-PO | 30 | 21 | 45 |
| 6.0% Methocel K100 | 60 | 32 | 52 |
| 6.0% Methocel K100M | 120 | 44 | 62 |
| 12.6% Xylit | 180 | 54 | 70 |
| 1.8% hydrocodone | 240 | 62 | 75 |
| 1% colloidal silicon dioxide | 300 | 68 | 80 |
| | 360 | 73 | 84 |
| | 420 | 78 | 87 |
| | 480 | 78 | 89 |

### EXAMPLE VI

### Pharmacokinetic Analysis of Formulations (Forms 26, 27, 28, and 29):

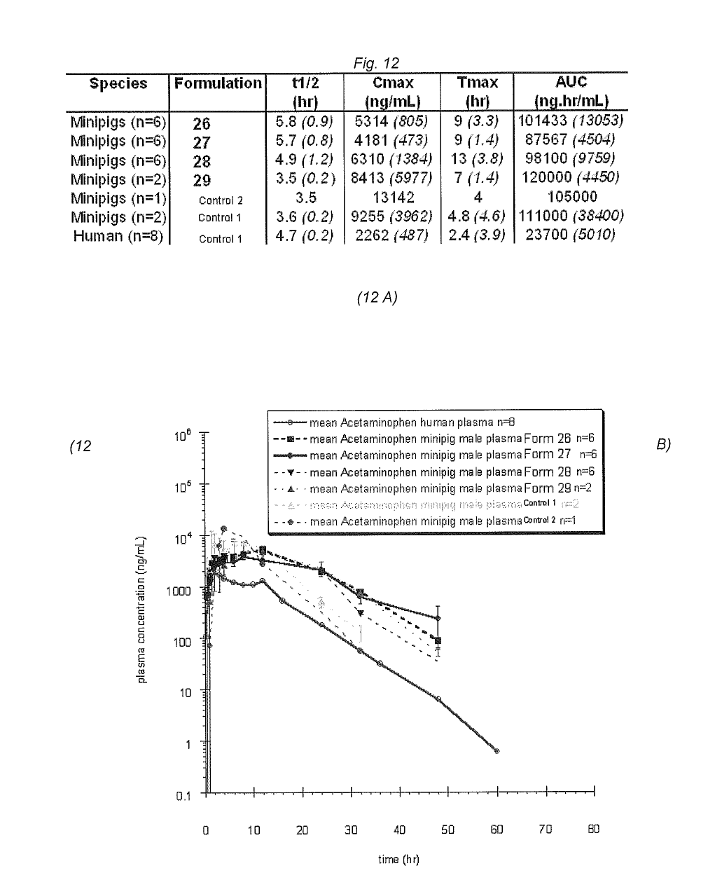

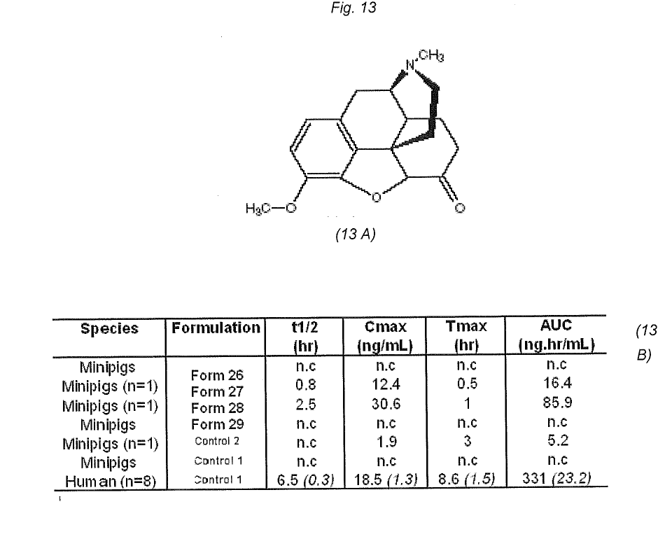

A set of exploratory studies were conducted to evaluate the bioequivalence of formulations of the invention (Forms 26-29), compared to a Control 1 formulation, which is similar to the formulation disclosed in Example 4 of Cruz et al. (U.S. Pat. Appln. Publn. No. 2005/0158382). The comparison of the PK profile of four inventive embodiments, one capsule formulation, and the Control 1 formulation after oral dose administration in male minipigs is demonstrated, also as shown in FIGS. 12 and 13. The PK profiles of these formulations are also compared with the PK profile of the Control 1 formulation from ALZA when dosed in Humans with normal liver functionality. The human data is collected from a separate study.

6 male Göttingen minipigs (11-15 kg; Ellegard, Denmark) used in these studies were subjected to oral dose administration with the formulations mentioned below in a randomized manner. The animals were fasted overnight prior to dosing but were permitted water ad libitum and food typically twelve hours post-dosing. Minipigs were housed individually in pens during the studies. For oral administration of tablets a balling gun was used followed by 50 mL of water. Before the dose administration a blood sample was taken from each animal.

### Forms 26-29 are shown below in Table 31:

TABLE 31

| | Formulation No. | | | | | |
|---|---|---|---|---|---|---|
| | Form 26 | Form 27 | Form 28 | Form 29 | Control 2 | Control 1 |
| Composition | 60% acetaminophen | 60% acetaminophen | 60% acetaminophen | 60% acetaminophen | | hydrocodone |
| | 11.4% Klucel EF | 13.6% Eudragit RL-PO | 10.1% Eudragit RL-PO | 12.6% Eudragit RL-PO | | 15 mg |
| | 11.4% Eudragit RL-PO | 13.6% Methocel | 6% Methocel K100 | 6% Methocel K100 | | acetaminophen |
| | 11.4% Methocel K100 | K100M | 6% Methocel K100M | 6% Methocel K100M | | 500 mg |
| | 3% Lutrol F68 | 10% Propylenglycole | 10.1% Klucel EF | 12.6% xylitol | | MMID D0500006 |
| | 1.8% hydrocodone | 1.8% hydrocodone | 5% Plurol Oleique CC | 1.8% hydrocodone | | |
| | 1% colloidal | 1% colloidal | 1.8% hydrocodone | 1% colloidal silicon | | |
| | silicon dioxide | silicon dioxide | 1% colloidal | dioxide | | |
| | | | silicon dioxide | | | |
| Target | 833.33 | 833.33 | 833.33 | 833.3 ( | 838.3 | 967.4 |
| weight | | | | | | |
| (mg) | | | | | | |

Potassium-ETDA blood samples were withdrawn from each animal at approximately 0, 0.5, 1.0, 1.5, 2, 3, 4, 6, 8, 12, 24, 32, 48 and 72 hours after drug administration. Upon collection, the samples were centrifuged at about 4° C. The resulting plasma samples were assayed for acetaminophen, hydrocodone and hydromorphone using a liquid chromatography—mass spectrometry method.

Observations:

Acetominophen plasma time profiles could be established for all formulations. Hydrocodone was detected after dosing of Forms 27 and 28 only. Signs of sedation was observed in all animals after dosing.

Acetaminophen Profile:

The half life observed in case of Form 26 (5.8 h) and Form 27 (5.9 h) formulations were similar. For Form 27 the t1/2 (half life) observed was 4.9 h. Whereas for Form 29 and Control 1 and Control 2 formulation indicated a similar half life of 3.5 h, 3.6 h and 3.5 h respectively and thus shorter than the other three formulations. Compared to the human Control 1 data the half life of the three forms (26, 27 & 28) were slightly longer but for Form 29, Control 2 and the Control 1 formulations have shorter half life.

As shown in FIGS. 12 and 13, the highest Cmax in minipigs was observed with Control 1 formulation. The Cmax observed with two minipigs with Control 1 formulation is 3 times higher than that observed with human. The Cmax for minipigs with Forms 26, 27, 28 & 29; Control 2 and Control 1 formulations were approximately 2-3 times higher than that observed in case of humans with Control 1 formulation.

The AUC in minipigs with Forms 26, 27, 28 & 29; Control 2 and Control 1 formulations were approximately 4 times higher than that observed in case of humans. The highest AUC in minipigs was observed with Form 29. The AUC (±sem) with Form 27 was 87567 (±4504) ng*h/ml, with Form 28 was 98100 (±9759) ng*h/ml, with Form 26 was 101433 (±13053) ng.h/ml and Form 29 was 120000 (±4450) ng*h/ml.

In all animals no acetaminophen was quantifiable in plasma after 48 hours of dose administration. A similar phenomenon was observed for humans except for one subject where the acetaminophen level in plasma was quantifiable till 60 h post-dose administration.

Hydrocodone and Hydromorphone Profile:

Hydrocodone was quantifiable in all human samples till 36 hours after dose administration. Whereas in case of minipigs no hydrocodone could be quantified above LOQ (1.2 ng/ml) in plasma except for two animals administered with three different formulations (Form 27 & 28 and Control 2).

In case of Form 28, the hydrocodone level could be quantified till 8 hours post-dose administration in one animal whereas in case of Form 27 with another animal, the hydrocodone level could be quantified till 3 hours post-dose administration. With Control 2 formulation the hydrocodone level was observed between 2 h and 4 h post-dose administration only. Only one animal showed hydrocodone levels with two different formulations, Form 27 and Control 2 formulation, on different days.

No hydromorphone was observed in either human or minipig plasma samples. These observations indicate species-specific hydrocodone metabolism compared to human. Intra-animal variation with respect to acetaminophen and hydrocodone plasma levels was observed.

### EXAMPLE VII

### Pharmacokinetic Analysis of Form 30:

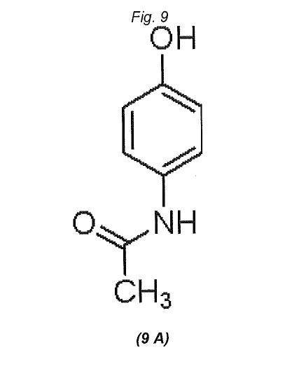

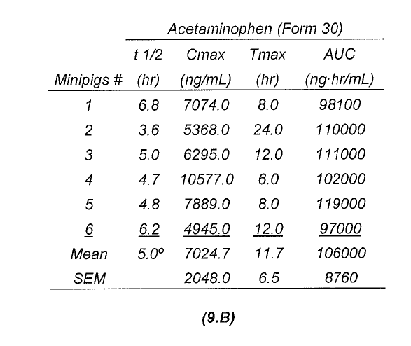

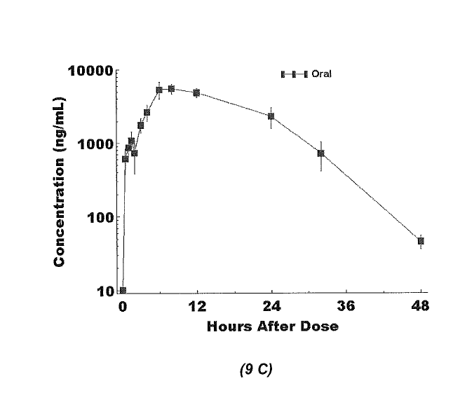

6 male Göttingen Minipigs (11-15 kg; Ellegard, Denmark) used in these studies were subjected to oral dose administration with Form 30, see Table 32. The animals were fasted overnight prior to dosing, but were permitted water ad libitum, and food typically twelve hours post-dosing. Minipigs were housed individually in pens during the studies. For oral administration of tablets a balling gun was used followed by 50 mL of water. Before the dose administration a blood sample was taken from each animal. Potassium-ETDA blood samples were withdrawn from each animal at approximately 0, 0.5, 1.0, 1.5, 2, 3, 4, 6, 8, 12, 24, 32, 48 and 72 hours after drug administration. Upon collection, the samples were centrifuged at about 4° C. The resulting plasma samples were assayed for acetaminophen using a liquid chromatography—mass spectrometry method, as shown in FIG. 9.

TABLE 32
Form 30

| Composition | 60% acetaminophen |
|---|---|
| | 11% Eudragit RL |
| | 11% Methocel K100M |
| | 12% Klucel EF |
| | 5% Cremophor EL |
| | 1% colloidal silicon dioxide |
| Target weight (mg) | 833.3 |

Observations: Acetominophen plasma time profiles were established for all animals.

The apparent terminal half life (t1/2) observed in case of Form 30 was 5.2 h. The Cmax was observed to be 7025 ng/ml and AUC 106000 ng*h/ml.

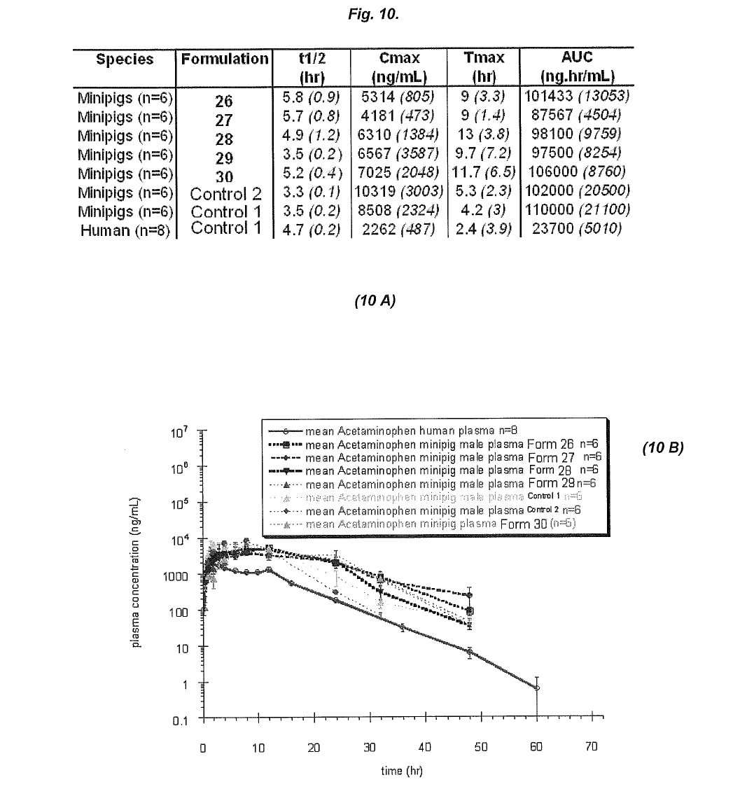

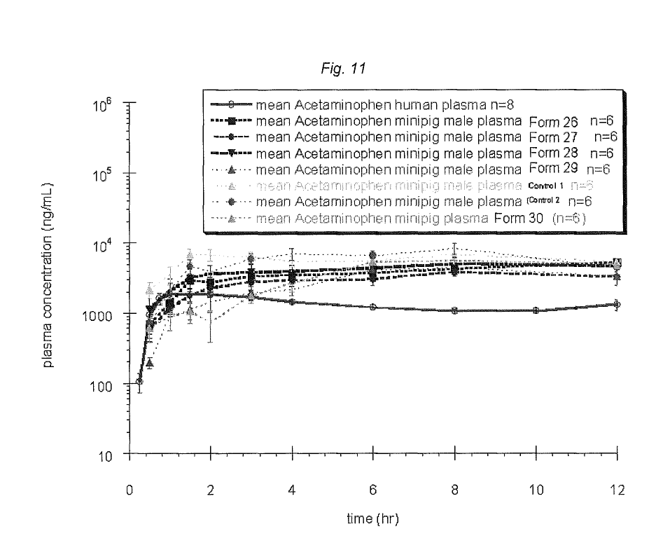

A comparison of the pharmacokinetic parameters obtained with Form 30 for minipigs, Control 1 and Control 2 formulations is demonstrated in FIGS. 10 and 11.

### EXAMPLE VIII

Certain exemplary abuse deterrent formulations were formulated on the basis of a combination of a retardation agent and a polymer which is insoluble or poorly-soluble in ethanol. The formulations listed below in Table 32 deter abuse of abuse relevant drugs (e.g., opioids) by making extraction of the drug of abuse more difficult. This is achieved by maintaining the controlled release characteristics of the formulation even after the dosage form is crushed and/or ground, and is preferably independent of the media. In the following examples and embodiments similar thereto, the rate of release after crushing or grinding in a coffee grinder (as defined hereinabove) preferably do not release drug at significantly increased rates, e.g., less than 40 percentage points faster, more preferably less than about 30 percentage points faster, and yet more preferably less than about 20 percentage points faster than the intact formulation in 0.01 N HCl or 20% or 40% aqueous ethanol, especially as measured from the time period of 1 to 4 hours after introduction into an aqueous medium or household solvent.

In certain exemplary preferred embodiments, components of the abuse deterrent formulations, include the following:

1. Eudragit RS or RL (ammonio methacrylate copolymer type B or type A) according to pharmacopoeas like e.g. USP/NF or Pharm. Eur.

2. polymer of category I-III (low solubility in EtOH, further defined below)

While any suitable mass ratios can be used, certain preferred ratio includes: Eudragit (RS, RL)/Polymer (I-III) 0.6 to 1.4:1, more preferably 0.8 to 1.2:1, and optionally about 1:1.

(a) Composition of certain formulations (by % weight) of the invention are defined by:

| | | |
|---|---|---|
| 1. | Active Pharmaceutical Ingredient: | up to 70% |
| 2. | Polymer A: Eudragit (RS, RL): | 20–80% |
| | Polymer B: Polymer of category | (sum of A + B) |
| | I–III from list below | |
| 3. | other excipients: | 0–25% |

(b) Shaping: In certain embodiments, a preferred method for shaping the tablets is calendering, however, any suitable method including, without limitation, direct shaping of the polymer melt (e.g., injection molding) can also be used. Milling and tabletting, on the other hand, is not a preferred alternative for shaping the tablets because it tends to lead to tablets that are more amenable to tampering (i.e., crushing or grinding so as to substantially degrade the controlled release profile of the formulation when exposed to a household solvent (as defined herein) or other aqueous solution.

(c) Certain polymers are used in the various formulations, based on the following categories, where: Category I reflects the most preferred polymers, Category II reflects the preferred polymers; category III reflects additional polymers useful in the context of the invention, and Category IV reflects polymers that can also be used, however, as additional excipients.

Some preferred formulations were based on solubility in aqueous ethanol, and thermoplastic properties of polymers, which may be necessary for use as base polymer in a melt extrusion process. Among these non-ionic polymers were preferred.

(d) Solubility in aqueous ethanol was based on the following criterion:

| Category | Solubility |
|---|---|
| I: | <3 Wt. % in H₂O/EtOH (80/20) |
| II: | 3 Wt. %–6 Wt. % in 20% aqueous ethanol |
| III: | 6 Wt. %–10 Wt. % in 20% aqueous ethanol |
| IV: | >10 Wt. % in 20% aqueous ethanol |

In the most preferred embodiment, preferred polymers should be thermoplasts with a solubility of less than 6 weight % 20% aqueous ethanol.

Certain exemplary abuse deterrent formulations are shown below in Table 33:

TABLE 33

| Polymer | Category | Substitution | Observations |
|---|---|---|---|
| Hydroxypropylcellulose (Klucel ®) | IV | Molecular substitution: | Water soluble; soluble |
| HF, ME, JF, | IV | 3.0 | in EtOH |
| LF, EF differ in viscosity | IV | | |
| | IV | | |
| | IV | | |
| Hydroxypropylcellulose | II or III | L-HPC | Low substitute, non- |
| | | | thermoplastic hydroxypropylcellulose |
| | | | (HPC) |
| Methylcellulose (Methocel ® A) | I | A: | Significantly less |
| | | —OMe 27.5–31.5% | soluble in EtOH than |
| | | | HPC |
| Methylcellulose | IV | —OMe 40–47% | |
| Hydroxyethylcellulose | III or II | | Water soluble, poor |
| | | | thermoplastic properties |
| Carboxymethylcellulose-Na | III or II | | Water soluble, poor |
| | | | thermoplastic properties |
| Ethylcellulose (Ethocel ®) | IV | Standard: | Medium: results in |
| | III or II | —OEt 48.0–49.5% | formation of gels |
| | | Medium: | |
| | | —OEt 45–47% | |
| Sodium Starch Glycolate | III or II | | Slightly soluble in |
| (Primojel ® | | | EtOH Insoluble in |
| | | | water |
| Starch | III or II | | Contains starch from |
| | | | corn, rice, potatoes |
| | | | and wheat |
| Gelatine | III or II | | Swells; soluble in hot |
| | | | water |
| Tragant | III or II | | 15–40% soluble in |
| | | | water formation of |
| | | | gels |
| Polyox | I or II | | Soluble in EtOH at >45° C., |
| Polyethylene Oxide NF | | | very good |
| | | | thermoplastic properties |
| Polyvinlypyrrolidon (PVP, | IV | | |
| Kollidon ®) | | | |
| Povidone USP (=PVP homopolymer) | | | |
| Copovidone Ph. Eur. (= PVP copolymer | | | |
| with vinyl-acetate) | | | |
| Polyethylenglycol (PEG) | IV | | |
| Polypropylenglycol (PPG) | IV | | |
| Eudragit | IV | L (methacrylic | Soluble in EtOH |
| Methacrylic acid copolymer, type | | acid copolymer | |
| A, NF (Eudragit ® L100) | | type A) S | |
| Methacrylic acid copolymer, type | | (methacrylic acid | |
| B, NF (Eudragit ® S100) | | copolymer type | |
| Methacrylic acid copolymer, type | | B) E (poly(butyl) | |
| C, NF (Eudragit ® L100-55) | | methacrylat | |
| Polyacrylate Dispersion 30 Percent | | NE30D | |
| Ph. Eur. = Eudragit NE30D | | (poly(ethylacrylat- | |
| (= 30% aqueous dispersion) | | methylmethacrylat)- | |
| Basic butylated methacrylate copolymer | | disperson | |
| Ph. Eur. = Eudragit E-100 | | | |
| Guar | III or II | | |
| Pectin | III or II | | |
| alginic acid/Na-alginate | III or II | | good thermoplastic |
| | | | properties |
| Arabic Gum | III or II | | |
| Hydroxypropyl methylcellulose | II or III | HPMCP | thermoplastic, ionic |
| phthalate | | | |
| Hypromellose Phthalate NF | | | |
| Hydroxypropyl-methylcellulose | II or III | AQOAT | thermoplastic, ionic |
| acetate phthalate | | | |
| Chitosan | II or III | | |
| Sodiumcarboxymethyl starch | III | Sodium Starch | not thermoplastic, |
| | | Glycolate | poorly soluble in |
| | | | EtOH |
| Polyvinyl-acetate | III | PVAC | thermoplastic, soluble |
| | | | in EtOH |
| Cellulose-Acetate | I–II | | thermoplastic, not- |
| Cellulose Acetat Butyrate | | | ionic, insoluble in |
| Cellulose Acetat Propionate | | | EtOH |

### EXAMPLE IX

### Relative Bioavailability of Form 45 Formulation Compared to Control 1 in Humans:

In this study the objective was to compare the relative bioavailability of a test formulation, Form 45 and reference Control 1.

Form 45 was manufactured as a tablet formulation for human clinical trials, as shown below: A homogeneous powder blend containing 1.8 kg acetaminophen, 54.0 g hydrocodone bitartrate pentahemihydrate, 378.0 g Eudragit® RL, 180.0 g Methocel® K100, 180.0 g Methocel® K100M, 378.0 g Xylitol and 29.9 g Colloidal silica (type: Aerosil® 200) was fed into an 6-barrel twin-screw extruder (screw diameter 18 mm) with a feeding rate of 1.5 kg/h. Rotation speed of the screws was 94 rpm and melt temperature was 140° C. The white homogeneous melt leaving the extruder at the die was directly shaped by a calendar having two counter-rotating rollers into elongated tablets. After cooling at room temperature the tablets were deburred in a container blender with high agitation in order to remove the seems on the tablet deriving from calendaring. The final tablets had a mean tablet weight of 833 mg according to a drug content of 500 mg (acetaminophen) and 15 mg (hydrocodone bitartrate pentahemihydrate) of each tablet.

The study was designed with the following parameters:

Single-dose, fasting, open-label, two-period, crossover study in 16 human subjects was carried out with the following regimens:

### Form 45: (1 tablet, 15 mg hydrocodone bitartrate/500 mg acetaminophen)

### Control 1: (1 tablet, 15 mg hydrocodone bitartrate/500 mg acetaminophen)

### Blood samples were collected at 0, 0.25, 0.5, 0.75, 1, 2, 3, 4, 6, 8, 10, 12, 16, 24, 36 and 48 hours after the dose on Study Day 1

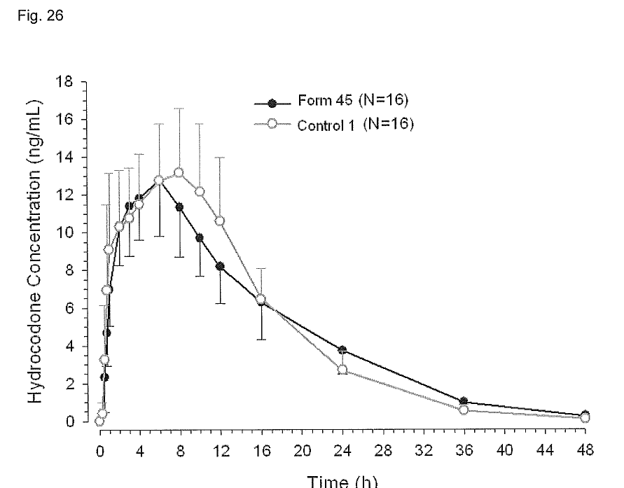

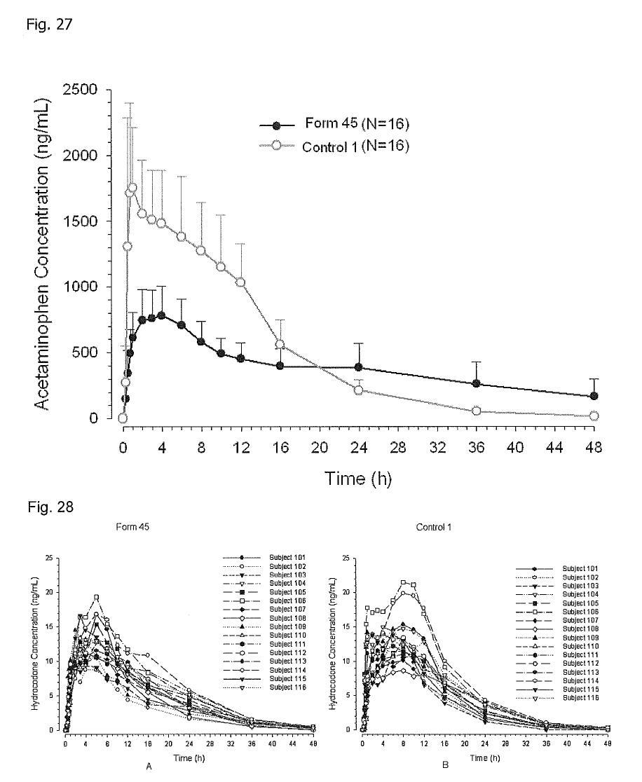

As shown in FIGS. 26 and 27 and in the following table 34, the preliminary pharmacokinetic indications are below for Form 45 vs. Control 1

Both Form 45 and Control 1 have similar Cₘₐₓ and AUC values for hydrocodone. However, for acetaminophen, Cmax is about 61% lower and AUCₜ is about 23% lower. Both Form 45 and Control 1 have similar AUC_{∞} for acetaminophen. For acetaminophen, apparent t1/2 for Form 45 is about 2-fold longer while Tmax is less variable.

Without ascribing to any particular theory the t1/2 value may be based on slow-release from Form 45 and tmax value may be based on the fact that Form 45 is not biphasic.

TABLE 34

| | Pharmacokinetic Parameters | | | | | |
|---|---|---|---|---|---|---|
| | | | AUCₜ | AUC_{inf} | | |
| Regimen | Tₘₐₓ (h) | Cₘₐₓ (ng/mL) | (ng * h/mL) | (ng * h/mL) | t_{1/2} (h) | CL/F (L/h) |
| | Hydrocodone | | | | | |
| Form 45 | 4.8 | 13.4 | 225 | 229 | 6.8 | 41.5 |
| | (33%) | (22%) | (22%) | (21%) | (16%) | (23%) |
| Control 1 | 6.8 | 13.6 | 225 | 229 | 5.5 | 41.7 |
| | (36%) | (25%) | (25%) | (24%) | (14%) | (22%) |
| | Acetaminophen | | | | | |
| Form 45 | 3.4 | 0.83 | 18.6 | 25.3 | 11.0 | 24.2 |
| | (37%) | (28%) | (29%) | (48%) | (71%) | (15%) |
| Control 1 | 2.3 | 2.12 | 24.1 | 24.3 | 5.8 | 21.8 |
| | (120%) | (24%) | (23%) | (23%) | (17%) | (27%) |

For the study in Example IX, additional pharmacokinetic details are provided in FIGS. 26-33.

FIG. 26 depicts mean hydrocodone concentration-time profiles for Form 45 and Control 1.

FIG. 27 depicts mean acetaminophen concentration-time profiles for Form 45 and Control 1.

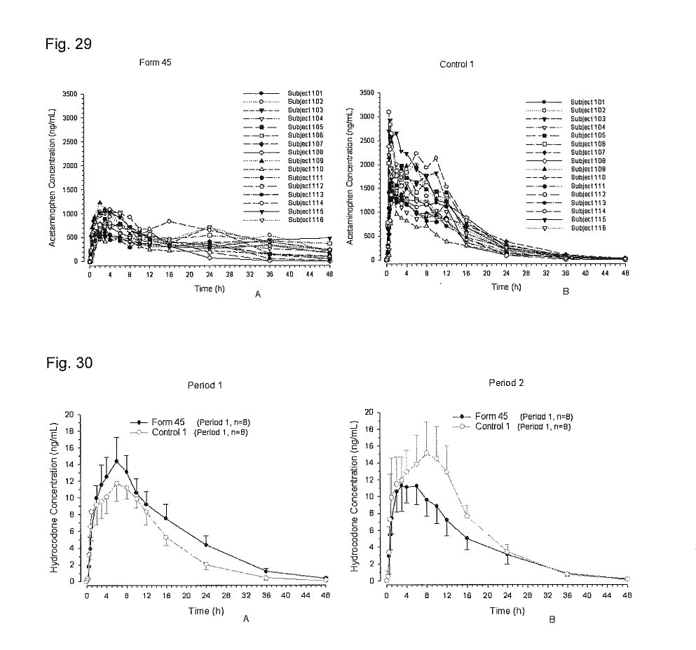

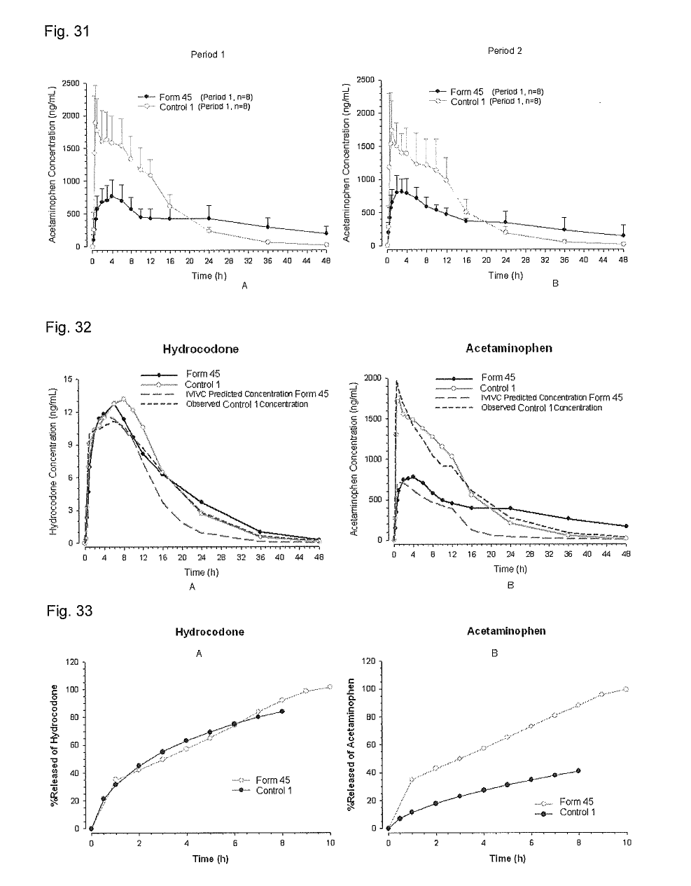

FIGS. 28 A and B depicts hydrocodone concentration-time profile for individual subject for Form 45 and Control 1, respectively. FIGS. 29 A and B depicts acetaminophen concentration-time profile for individual subject for Form 45 and Control 1, respectively. FIGS. 30 A and B depicts mean hydrocodone concentration-time profile for period 1 and 2, respectively for Form 45 and Control 1. FIGS. 31 A and B depicts mean acetaminophen concentration-time profile by periods 1 and 2, respectively for Form 45 and Control 1. FIGS. 32 A and B depicts mean hydrocodone and acetaminophen concentrations for in vitro Form 45, in vitro Control 1, in vivo Control 1 concentration and in vitro-in vivo concentration predictions for Form 45. FIGS. 33 A and B depicts mean hydrocodone and acetaminophen in vitro dissolution profiles for Form 45 and Control 1. FIG. 26 depicts mean hydrocodone concentration-time profiles for Form 45 and Control 1.

The foregoing detailed description and accompanying examples are merely illustrative and not intended as limitations upon the scope of the invention, which is defined solely by the appended claims and their equivalents. Various changes and modifications to the disclosed embodiments will be apparent to those skilled in the art and are part of the present invention. Such changes and modifications, including without limitation those relating to the chemical structures, substituents, derivatives, intermediates, syntheses, formulations and/or methods of use of the invention, can be made without departing from the spirit and scope thereof.

What is claimed is:

1. An abuse-deterrent drug formulation comprising a melt-processed mixture of
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

2. The formulation of claim 1, wherein the cellulose ether is hydroxpropyl methylcellulose.

3. The formulation of claim 1, wherein the alkyl alkacrylate or the alkacrylate polymer has monomeric units of (C₁-C₂₂)alkyl ((C₁-C₁₀)alk)acrylate or (C₁-C₁₀)alkacrylate.

4. The formulation of claim 1, wherein the alkacrylate polymer is an acrylic polymer or a methacrylic polymer.

5. The formulation of claim 1, wherein the alkacrylate polymer is ionic acrylic polymer or ionic methacrylic polymer.

6. The formulation of claim 1, wherein the alkacrylate polymer is a cationic acrylic polymer or cationic methacrylic polymer.

7. The formulation of claim 1, wherein the alkacrylate polymer is a copolymer of the acrylic polymer and the methacrylic polymer esters containing quaternary ammonium groups.

8. The formulation of claim 1, wherein the abuse-relevant drug is selected from the group consisting of atropine, hyoscyamine, phenobarbital, and scopolamine salts, esters, prodrugs and mixtures thereof.

9. The formulation of claim 1, wherein the abuse-relevant drug is an analgesic.

10. The formulation of claim 1, wherein the abuse-relevant drug is an opioid.

11. The formulation as claimed in claim 10, wherein the opioid is selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levophenacylmorphan, levorphanol, lofentanil, meperidine, meptazinol, metazocine, methadone, metopon, morphine, myrophine, nalbulphine, narceine, nicomorphine, norpipanone, opium, oxycodone, oxymorphone, papvretum, pentazocine, phenadoxone, phenazocine, phenomorphan, phenoperidine, piminodine, propiram, propoxyphene, sufentanil, tilidine, and tramadol, and salts, esters, prodrugs and mixtures thereof.

12. The formulation as claimed in one of claims 8-11, further comprising at least one further drug.

13. The formulation of claim 1, wherein the abuse-relevant drug is dispersed in the formulation in a state of a solid solution.

14. The formulation of claim 1, wherein between 11% and 47% of the abuse-relevant drug is released in vitro in 0.01 N hydrochloric acid within two hours at 37° C.

15. The formulation of claim 1, wherein less than 20% of the abuse-relevant drug is released in vitro in 20% aqueous ethanol within one hour at 37° C.

16. The formulation of claim 1, wherein the dosage form is monolithic

17. A monolithic, sustained release oral dosage formulation comprising a melt-processed mixture of:
a) an analgesically effective amount of at least one an abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted for sustained release so as to be useful for oral administration to a human 3, 2, or 1 times daily.

18. The formulation of claim 17, wherein the cellulose ether is hydroxpropyl methylcellulose.

19. The formulation of claim 17, wherein the alkacrylate polymer is an acrylic polymer or a methacrylic polymer.

20. The formulation of claim 17, wherein the alkacrylate polymer is an ionic acrylic polymer or an ionic methacrylic polymer.

21. The formulation of claim 17, wherein the alkacrylate polymer is a cationic acrylic polymer or a cationic methacrylic polymer.

22. The formulation of claim 17, wherein the alkacrylate polymer is a copolymer of the acrylic polymer and the methacrylic polymer esters containing quaternary ammonium groups.

23. The formulation of claim 17, wherein the abuse-relevant drug is an analgesic.

24. The formulation of claim 17, wherein the abuse-relevant drug is an opioid.

25. The formulation as claimed in one of claims 23-24 further comprising at least one further drug.

26. The formulation of claim 17, wherein the abuse-relevant drug is dispersed in the formulation in a state of a solid solution.

27. The formulation of claim 17, wherein between 11% and 47% of the abuse-relevant drug is released in vitro in 0.01 N hydrochloric acid within two hours at 37° C.

28. The formulation of claim 17, wherein less than 20% of the abuse-relevant drug is released in vitro in 20% aqueous ethanol within one hour at 37° C.

29. An oral sustained release dosage formulation of a drug characterized by at least two of the following features:
a) the drug that is extracted from the formulation by 40% aqueous ethanol within one hour at 37° C. in vitro is less than or equal twice the amount of the drug that is extracted by 0.01 N hydrochloric acid in vitro within one hour at 37° C.,
b) the formulation does not break under a force of 150 newtons, preferably 300 newtons, more preferably 450 newtons, yet more preferably 500 newtons as measured by “Pharma Test PTB 501” hardness tester, and
c) the formulation releases at least 15% of the one drug and not more than 45% of the one drug during the first hour in vitro dissolution testing and preferably also in vivo.

30. The oral sustained release dosage formulation of claim 29, wherein the formulation is not snortable via nasal administration.

31. The oral sustained release dosage formulation of claim 29, wherein the drug is an opioid, amphetamine or methamphetamine.

32. The oral sustained release dosage formulation of claim 29, wherein the formulation comprises an abuse-deterrent drug produced by a melt-processed mixture of
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid in vitro within one hour at 37° C.; and
wherein the drug formulation is adapted so as to be useful for oral administration to a human 3, 2, or 1 times daily.

33. The oral sustained release dosage formulation of claim 32, wherein the cellulose ether is hydroxpropyl methylcellulose.

34. The oral sustained release dosage formulation of claim 32, wherein the alkyl alkacrylate or the alkacrylate polymer has monomeric units of (C₁-C₂₂)alkyl ((C₁-C₁₀)alk)acrylate or (C₁-C₁₀)alkacrylate.

35. The oral sustained release dosage formulation of claim 32, wherein the alkacrylate polymer is an acrylic polymer or a methacrylic polymer.

36. The oral sustained release dosage formulation of claim 32, wherein the alkacrylate polymer is ionic acrylic polymer or ionic methacrylic polymer.

37. The oral sustained release dosage formulation of claim 32, wherein the alkacrylate polymer is a cationic acrylic polymer or cationic methacrylic polymer.

38. The oral sustained release dosage formulation of claim 32, wherein the alkacrylate polymer is a copolymer of the acrylic polymer and the methacrylic polymer esters containing quaternary ammonium groups.

39. The oral sustained release dosage formulation of claim 32, wherein the alkacrylate polymer is a copolymer or mixture of copolymers wherein the molar ratio of cationic groups to the neutral esters is in the range of about 1:20 to 1:35 on average.

40. A non-milled, melt-extruded drug formulation comprising a drug with abuse potential.

41. The formulation of claim 40, wherein the formulation is not snortable via nasal administration.

42. The formulation of claim 40, wherein the drug is an opioid, amphetamine or methamphetamine.

43. The formulation of claim 40, wherein the formulation is directly shaped from the melt-extrudate into a dosage form without (an intermediate) milling step.

44. The formulation of claim 40, wherein the formulation is directly shaped from the melt-extrudate into a dosage form without (an intermediate) multiparticulating step.

45. The formulation of claim 40, wherein the formulation is directly shaped from the melt-extrudate into a dosage form by the process of calendaring.

46. A monolithic, non-milled, non-multiparticulated, melt-extruded drug formulation comprising a drug with abuse potential having a diameter from about at least 5.1 mm to about 10 mm and a length from about 5.1 mm to about 30 mm.

47. The formulation of claim 46, wherein the formulation is directly shaped from the melt-extrudate into a dosage form without (an intermediate) milling step.

48. The formulation of claim 46, wherein the formulation is directly shaped from the melt-extrudate into a dosage form without (an intermediate) multiparticulating step.

49. The formulation of any of the claims 46-48 wherein the formulation is directly shaped from the melt-extrudate into a dosage form by the process of calendaring.

50. The formulation of claim 46, wherein the formulation comprises an abuse-deterrent drug produced by a melt-processed mixture of
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

51. The formulation of claim 50, wherein the alkacrylate polymer is a copolymer of the acrylic polymer and the methacrylic polymer esters containing quaternary ammonium groups.

52. An abuse-deterrent drug formulation formed by a process comprising melt extruding the formulation having at least one therapeutic drug and directly shaping the extrudate into a dosage form without (an intermediate) milling step or multiparticulating step.

53. The formulation of claim 52, wherein the therapeutic drug comprises an abuse-deterrent drug having:
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

54. A process for the manufacture of an abuse-resistant drug dosage formulation comprising melt extruding a formulation comprising at least one therapeutic drug further comprising directly shaping the extrudate into a dosage form without (an intermediate) milling step or multiparticulating step.

55. The process of claim 54, wherein the melt-extrudate comprises an abuse-deterrent drug having:
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

56. A monolithic, non-milled, melt-extruded drug formulation comprising a drug with abuse potential wherein the monolithic formulation has a substantially similar drug release profile to a crushed form of the monolithic formulation wherein the monolithic formulation is crushed at about 20,000 rpm to about 50,000 rpm in a coffee grinding machine for about 60 seconds.

57. The melt-extrudate drug formulation of claim 56, wherein the melt-extrudate comprises an abuse-deterrent drug having:
a) at least one abuse-relevant drug,
b) at least one cellulose ether or cellulose ester, and
c) at least one alkyl alkacrylate polymer, alkacrylate polymer, or a combination thereof,
wherein the amount of the drug that is extracted in vitro from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

58. The melt-extrudate drug formulation of claim 57, wherein the drug formulation does not comprise more than 0.5% of a genotoxic compound after manufacturing and a minimum of 6 months of storage at 25° C./60% relative humidity or 40° C./75% relative humidity, or both.

59. The melt-extrudate drug formulation of claim 58, wherein the formulation comprises polyethylene oxide and an anti-oxidant.

60. The melt-extrudate drug formulation of claim 58, wherein the genotoxic compound is N-oxide of an opioid.

61. An abuse-deterrent drug formulation comprising a melt-processed mixture of
at least one abuse-relevant drug, and
at least one rate altering pharmaceutically acceptable polymer, copolymer, or a combination thereof,
wherein the amount of the drug that is extracted from the formulation by 40% aqueous ethanol within one hour at 37° C. is less than or equal to twice the amount of the drug that is extracted by 0.01 N hydrochloric acid within one hour at 37° C.; and
wherein the drug formulation is adapted so as to be useful for oral administration to a human 3, 2, or 1 times daily.

62. The abuse-deterrent drug formulation of claim 61, wherein the polymer is a cellulose ether or a cellulose ester polymer.

63. The abuse-deterrent drug formulation of claim 61, wherein the polymer is selected from a group consisting of homopolymers, copolymers, or combinations of monomers of N-vinyl lactams, nitrogen-containing monomers, oxygen-containing monomers, vinyl alcohol, ethylene glycol, alkylene oxides, ethylene oxide, propylene oxide, acrylamide, vinyl acetate, hydroxy acid.

64. The abuse-deterrent drug formulation of claim 61, wherein the polymer is hydrogen-peroxide polyvinylpyrrolidone polymer.

65. The abuse-deterrent drug formulation of claim 61, wherein the polymer, copolymer, or a combination thereof comprises at least one alkyl alkacrylate polymer, alkacrylate polymer, or a combination thereof.

66. The abuse-deterrent drug formulation of claim 62, wherein the cellulose ether has an alkyl degree of substitution of 1.3 to 2.0 and hydroxyalkyl molar substitution of up to 0.85.

67. The abuse-deterrent drug formulation of claim 66, wherein the alkyl substitution is methyl.

68. The abuse-deterrent drug formulation of claim 67, wherein the hydroxyalkyl substitution is hydroxpropyl.

69. The abuse-deterrent drug formulation of claim 62, wherein the cellulose ether is hydroxpropyl methylcellulose.

70. The abuse-deterrent drug formulation of claim 61, wherein the alkyl alkacrylate or the alkacrylate polymer has monomeric units of (C₁-C₂₂)alkyl ((C₁-C₁₀)alk)acrylate or (C₁-C₁₀)alkacrylate.

71. The abuse-deterrent drug formulation of claim 61, wherein the alkacrylate polymer is an acrylic polymer or a methacrylic polymer.

72. The abuse-deterrent drug formulation of claim 61, wherein the alkacrylate polymer is ionic acrylic polymer or ionic methacrylic polymer.

73. The abuse-deterrent drug formulation of claim 61, wherein the alkacrylate polymer is a cationic acrylic polymer or cationic methacrylic polymer.

74. The abuse-deterrent drug formulation of claim 61, wherein the alkacrylate polymer is a copolymer of the acrylic polymer and the methacrylic polymer esters containing quaternary ammonium groups.

75. The abuse-deterrent drug formulation of claim 61, wherein the alkacrylate polymer is a copolymer or mixture of copolymers wherein the molar ratio of cationic groups to the neutral esters is in the range of about 1:20 to 1:35 on average.

76. An abuse-deterrent drug formulation comprising a melt-processed mixture of
a) at least one abuse-relevant drug, wherein said drug is hydrocodone,
b) at least one cellulose ether or cellulose ester, and
c) at least one acrylic polymer, methacrylic polymer, or a combination thereof,
wherein the drug formulation is adapted so as to be useful for oral administration to a human 3, 2, or 1 times daily; and
wherein about 90% of the hydrocodone is released in vitro at about 4-6 hours when adapted to be administered 3 times a day, at about 6-10 hours when adapted to be administered 2 times a day and about 16-22 hours when adapted to be administered 1 time a day.

77. The abuse-deterrent drug formulation of claim 76, wherein more than 30% of the hydrocodone is extracted from the formulation at about one hour at 37° C. in 0.01 N hydrochloric acid.

78. The abuse-deterrent drug formulation of claim 76, wherein from about 12% to about 25% of the hydrocodone is extracted from the formulation at about one hour at 37° C. in 0.01 N hydrochloric acid.

79. An abuse-deterrent drug formulation comprising a melt-processed mixture of
at least one opioid;
at least one rate altering pharmaceutically acceptable polymer, copolymer, or a combination thereof;
wherein the amount of the drug that is extracted from the formulation by 40% aqueous ethanol within one hour at 37° C. is about 70% to about 110% of the amount of the drug that is extracted by 0.01 N hydrochloric acid within one hour at 37° C.; and
wherein the drug formulation is adapted so as to be useful for oral administration to a human 3, 2, or 1 times daily.

80. The abuse-deterrent drug formulation of claim 79, wherein the amount of the drug that is extracted from the formulation by 40% aqueous ethanol within one hour at 37° C. is about 70% to about 100% of the amount of the drug that is extracted by 0.01 N hydrochloric acid within one hour at 37° C.

81. The abuse-deterrent drug formulation of claim 79, wherein the amount of the drug that is extracted from the formulation by 40% aqueous ethanol within one hour at 37° C. is about 70% to about 90% of the amount of the drug that is extracted by 0.01 N hydrochloric acid within one hour at 37° C.

82. The abuse-deterrent drug formulation of claim 79, wherein the amount of the drug that is extracted from the formulation by 40% aqueous ethanol within one hour at 37° C. is about 75% to about 90% of the amount of the drug that is extracted by 0.01 N hydrochloric acid within one hour at 37° C.

83. The abuse-deterrent drug formulation of claim 79, wherein the abuse relevant drug further comprises a nonopioid analgesic.

84. The abuse-deterrent drug formulation of claim 79, wherein the non-opioid analgesic is acetaminophen or ibuprofen.

85. The abuse-deterrent drug formulation of claim 79, wherein the opioid is hydrocodone or oxycodone, or pharmaceutically acceptable salts or esters thereof.

86. The abuse-deterrent drug formulation of claim 79, wherein the opioid is hydrocodone and wherein when administered to the human patient, the formulation produces a plasma profile characterized by a Cmax for hydrocodone of between about 0.6 ng/mL/mg to about 1.4 ng/mL/mg after a single dose.

87. The abuse-deterrent drug formulation of claim 79, wherein the opioid is hydrocodone and wherein when administered to the human patient, the formulation produces a plasma profile characterized by a Cmax for hydrocodone of between about 0.4 ng/mL/mg to about 1.9 ng/mL/mg after a single dose.

88. The abuse-deterrent drug formulation of claim 79, wherein the opioid is hydrocodone and wherein when administered to the human patient, the formulation produces a plasma profile characterized by a Cmax for hydrocodone of form about 0.6 ng/mL/mg to about 1.0 ng/mL/mg after a single dose.

89. The abuse-deterrent drug formulation of claim 79, wherein the opioid is hydrocodone and wherein when administered to the human patient, the formulation produces a plasma profile characterized by a Cmin for hydrocodone of between about 0.4 ng/mL/mg, or optionally 0.6 ng/mL/mg, to about 1.4 ng/mL/mg after a single dose.

90. The abuse-deterrent drug formulation of claim 79, wherein the opioid is hydrocodone and wherein when administered to the human patient, the dosage form produces a minimum AUC for hydrocodone of about 7.0 ng*hr/mL/mg to a maximum AUC for hydrocodone of about 26.2 ng*hr/mL/mg.

91. The abuse-deterrent drug formulation of claim 79, wherein the opioid is hydrocodone and wherein when administered to the human patient, the dosage form produces a minimum AUC for hydrocodone of about 9.1 ng*hr/mL/mg to a maximum AUC for hydrocodone of about 19.9 ng*hr/mL/mg

92. The abuse-deterrent drug formulation of claim 79, wherein the in vitro rate of release of the formulation has a biphasic release profile, and wherein each phase of the in vitro rate of release is zero order or ascending.

93. The abuse-deterrent drug formulation of claim 79, wherein at least 30-45% of the opioid is released in vitro from the formulations in about 1 hour.

94. The abuse-deterrent drug formulation of claim 79, wherein at least 90% is of the opioid is released from the formulation in about 6 hours to about 10 hours.

95. The abuse-deterrent drug formulation of claim 79, wherein at least 90% is of the opioid is released from the formulation in about 15 hours to about 20 hours.

96. The abuse-deterrent drug formulation of claim 79, wherein at least 90% is of the opioid is released from the formulation in about 6 hours to about 9 hours.

97. The abuse-deterrent drug formulation of claim 79, wherein at least 95% is of the opioid is released from the formulation in about 6 hours to about 10 hours, and
wherein at least 95% is of the opioid is released from the formulation in about 7 hours to about 9 hours.

98. The abuse-deterrent drug formulation of claim 79, wherein at least 99% is of the opioid is released from the formulation in about 10 hours to about 11 hours.

99. The abuse-deterrent drug formulation of claim 79, wherein at least 99% is of the opioid is released from the formulation in less than about 12 hours.

100. The abuse-deterrent drug formulation of claim 79, wherein the AUC at one hour is from 0.22 to about 0.51 ng*h/ml/mg.

101. The abuse-deterrent drug formulation of claim 79, wherein the AUC at two hour is from 1.07 to about 1.76 ng*h/ml/mg.

102. The abuse-deterrent drug formulation of claim 79, wherein the AUC at three hour is from 2.06 to about 3.08 ng*h/ml/mg.

103. The abuse-deterrent drug formulation of claim 79, wherein the AUC at four hour is from 3.12 to about 4.44 ng*h/ml/mg.

104. A method for treating pain in a human patient, comprising orally administering to the human patient a formulation from any one of the claim 1-103.
